(19) United States
(12) Patent Application Publication
(10) Pub. No.: US 2026/0152793 A1
(43) Pub. Date: Jun. 4, 2026

(54) NOVEL EXPRESSION BASED, NON-INVASIVE METHOD TO DIFFERENTIATE ATOPIC DERMATITIS AND PSORIASIS

(71) Applicant: DERMTECH, INC, San Diego, CA (US)

(72) Inventors: Michael HOWELL, La Jolla, CA (US); John W. WHITAKER, La Jolla, CA (US)

(21) Appl. No.: 18/865,624

(22) PCT Filed: May 12, 2023
(86) PCT No.: PCT/US2023/066973

§ 371 Date: Nov. 13, 2024

Related U.S. Application Data
(60) Provisional application No. 63/341,963, filed on May 13, 2022.

(51) Int. Cl. C12Q 1/6883; A61B 10/02; G16B 25/10; G16B 40/20; G16H 50/20

(52) U.S. Cl. CPC C12Q 1/6883; A61B 10/02; G16B 25/10; G16B 40/20; A61B 2560/0412; C12Q 2600/112; G16H 50/20

(57) ABSTRACT

Provided herein are systems and methods for differentiating atopic dermatitis from psoriasis. Further provided herein are gene expression ratios for differentiating atopic dermatitis from psoriasis. Further provided herein are non-invasive methods for differentiating atopic dermatitis from psoriasis.

### CROSS-REFERENCE

This application claims the benefit of U.S. Provisional Application No. 63/341,963 filed May 13, 2022, which is incorporated herein by reference in its entirety.

### BACKGROUND OF THE DISCLOSURE

Skin diseases are some of the most common human illnesses and represent an important global burden in healthcare. Three skin diseases are in the top ten most prevalent diseases worldwide, and eight fall into the top 50. When considered collectively, skin conditions range from being the second to the 11th leading causes of years lived with disability.

### SUMMARY OF THE DISCLOSURE

An aspect of the disclosure herein describes methods of determining if a subject has atopic dermatitis or psoriasis, comprising: receiving a plurality of expression levels, wherein the expression levels are derived from a skin sample obtained using a non-invasive or semi-invasive technique; calculating a ratio between at least two of the plurality of expression levels; determining whether the ratio is above or below an indicator value; and generating an indication, wherein: the indication indicates that the subject has a first condition if the ratio is above the indicator value, the indication indicates that the subject has a second condition if the ratio is below the indicator value, the first condition is different from the second condition, the first condition is atopic dermatitis or psoriasis, and the second condition is atopic dermatitis or psoriasis. In some embodiments, the plurality of expression levels are determined by: obtaining the skin sample; isolating nucleic acids from the skin sample; and measuring the plurality of expression levels. In some embodiments, the plurality of expression levels are associated with a plurality of genes. In some embodiments, the plurality of genes comprises IL13, IL4R, CCL17, CCL26, IL23A, IL22, S100A7, S100A8, S100A9, CXCL9, CXCL10, CXCL11, NOS2, IL17A, IL13R, CCL19, CCL27, TSLP, CCL18, IL23A, IL4R, and IL31. In some embodiments, the plurality of genes comprises IL13, CCL17, NOS2, and IL17A. In some embodiments, the ratio is determined by dividing a sum of an expression level of IL13 and an expression level of CCL17 by a sum of an expression level of NOS2 and an expression level of IL17A. In some embodiments, calculating the ratio between the at least two of the plurality of expression levels is performed by a machine learning model, and determining whether the ratio is above or below the indicator value is performed by the machine learning model. In some embodiments, the indicator value is 1. In some embodiments, an AUC for the indicator value is at least 0.94. In some embodiments, the AUC for the indicator value is at least 0.83. In some embodiments, the method further comprises assigning a classifier to at least one expression level of the plurality of expression levels. In some embodiments, generating the indication is further based on the classifier. In some embodiments, the non-invasive technique comprises use of one or more adhesive patches or microneedles. In some embodiments, use of the one or more adhesive patches collects at least 1.5 mg of skin cells. In some embodiments, the skin sample comprises skin cells from the stratum corneum. In some embodiments, the skin sample is obtained from a lesional area of the subject. In some embodiments, the skin sample is stable for up to 10 days. In some embodiments, the skin sample is stored at 10-30 degrees C. In some embodiments, the isolated nucleic acids comprise DNA and/or RNA. In some embodiments, the skin sample comprises one or more of epithelial acanthosis and mononuclear perivascular infiltrates. In some embodiments, the isolated nucleic acids are amplified prior to measuring biomarkers. In some embodiments, the measuring comprises detecting the plurality of expression levels of one or more target genes by measuring one or more biomarkers. In some embodiments, the detecting gene expression levels comprises hybridizing one or more probes to nucleic acids corresponding to the one or more target genes. In some embodiments, the detecting gene expression levels comprises mass tagging and/or qPCR. In some embodiments, the one or more biomarkers comprises 2 or more target genes. In some embodiments, the one or more biomarkers comprises 5 or more target genes. In some embodiments, the one or more biomarkers comprises no more than 50 target genes. In some embodiments, the one or more target genes is differentially expressed in atopic dermatitis vs. psoriasis.

Provided herein are methods useful for differentiating inflammatory skin diseases comprising: obtaining a sample, wherein the sample is obtained using a non-invasive technique from a subject; isolating nucleic acids from the sample; measuring one or more biomarkers from the isolated nucleic acids; applying an algorithm to the one or more biomarkers to generate a pathology score; and identifying the sample as having atopic dermatitis or psoriasis based on the pathology score. Further provided are methods wherein the non-invasive or minimally-invasive technique comprises use of one or more adhesive patches or microneedles, respectively. Further provided are methods wherein the sample comprises skin cells from the stratum corneum or blood. Further provided are methods wherein the sample comprises one or more of epidermal keratinocytes, T cells, dendritic cells, and melanocytes. Further provided are methods wherein use of the one or more adhesive patches collects at least 1.5 mg of skin cells. Further provided are methods wherein the sample is obtained from a lesional area of the subject. Further provided are methods wherein the sample is obtained from a non-lesional area of the subject. Further provided are methods where the sample is obtained from a subject who is asymptomatic. Further provided are methods where the sample is obtained from a subject who is symptomatic. Further provided are methods wherein the sample is stable for up to 10 days. Further provided are methods wherein the sample is stored at 10-30 degrees C. Further provided are methods wherein the isolated nucleic acids comprise DNA and/or RNA. Further provided are methods wherein the sample comprises one or more of epithelial acanthosis and mononuclear perivascular infiltrates. Further provided are methods wherein the isolated nucleic acids are amplified prior to measuring biomarkers. Further provided are methods wherein the measuring comprises detecting gene expression levels of one or more target genes. Further provided are methods wherein the detecting gene expression levels comprises hybridizing one or more probes to nucleic acids corresponding to the one or more target genes. Further provided are methods wherein the detecting gene expression levels comprises mass tagging and/or qPCR. Further provided are methods wherein the one or more biomarkers comprises 2 or more target genes. Further provided are methods wherein the one or more biomarkers comprises 5 or more target genes. Further provided are methods wherein the one or more biomarkers comprises no more than 50 target genes. Further provided are methods wherein the one or more target genes is differentially expressed in atopic dermatitis vs. psoriasis. Further provided are methods wherein the one or more target genes are selected from the group consisting of TNF, LINC02571, HLA-C, HCP5, LCE3A-E, PSORS1C1, IFNG, IL12B, IL1B, NFKB1A, MUC22, and IL36RN. Further provided are methods wherein the one or more target genes are selected from the group consisting of IL4-R, FLG, SPINK5, EMSY, PBX2, FLG-AS1, TSBP1, CRCT1, STAT3, CLDN1, NLRP10, IL18R1, TNFRSF6B, TNXB, TSLP/R, and STAT6. Further provided are methods wherein the one or more target genes are selected from the group consisting of IL-13, IL-23, IL-17A, S100A8, S100A9, CXCL9, CXCL10, CCL17 (TARC), CCL18 (PARC), CCL27 (Eotaxin-3), TLSP, and NOS2. Further provided are methods wherein the one or more target genes are selected from the group consisting of HLA-B, KPNA3, MGMT, R3GCC1L, STEAP-AS2, PRR5L, and IL-13. Further provided are methods wherein the one or more target genes are selected from the group consisting of IL-4R, IL-13, CCL17/TARC, CCL26/Eotaxin 3, IL-17A, IL-22, IL-23A, NOS2, S100A8, S100A9, CXCL9, and CXCL10. Further provided are methods wherein the one or more target genes are selected from the group consisting of IL-4R, IL-13, CCL17/TARC, and CCL26/Eotaxin 3. Further provided are methods wherein the one or more target genes are selected from the group consisting of IL-17A, IL-22, IL-23A, NOS2, S100A8, S100A9, CXCL9, and CXCL10. Further provided are methods wherein the one or more target genes are selected from the group consisting of IL-13, CCL17/TARC, NOS2, and IL-17A. Further provided are methods wherein the one or more target genes are selected from a group consisting of TNF, LINC02571, HLA-C, HCP5, LCE3A-E, PSORS1C1, IFNG, IL12B, IL1B, NFKB1A, MUC22, IL36RN, IL4-R, FLG, SPINK5, EMSY, PBX2, FLG-AS1, TSBP1, CRCT1, STAT3, CLDN1, NLRP10, IL18R1, TNFRSF6B, TNXB, TSLP/R, STAT6, IL-13, IL-23, IL-17A, S100A8, S100A9, CXCL9, CXCL10, CCL18 (PARC), CCL27 (Eotaxin-3), TLSP, NOS2, HLA-B, KPNA3, MGMT, R3GCC1L, STEAP-AS2, PRR5L, IL-4R, IL-13, CCL17/TARC, CCL26/Eotaxin 3, IL-22, IL-23A, NOS2, S100A8, S100A9, IL-22, IL-23A. Further provided are methods wherein the algorithm comprises a ratio comparison of expression levels of two or more target genes. Further provided are methods wherein the algorithm comprises a ratio comparison of expression levels of four or more target genes. Further provided are methods wherein the algorithm comprises a ratio comparison of expression levels of no more than 50 target genes. Further provided are methods wherein the algorithm comprises a ratio comparison of CCL17+IL-13 expression levels divided by NOS2+IL-17A expression levels. Further provided are methods wherein a pathology score greater than 1 is indicative of atopic dermatitis in the sample. Further provided are methods wherein a pathology score less than 1 is indicative of psoriasis in the sample. Further provided are methods wherein the method comprises calculating an AUC. Further provided are methods, wherein the AUC is at least 0.90. Further provided are methods wherein the AUC is at least 0.94. Further provided are methods wherein the method comprises calculating an AUC. Further provided are methods wherein the AUC is at least 0.94 and the p-value is less than 0.001. Further provided are methods wherein the AUC is at least 0.94 and the p-value is less than 0.0001. Further provided are methods wherein the identifying the sample as having atopic dermatitis is based a pathology score >0.9. Further provided are methods wherein the identifying the sample as having psoriasis is based a pathology score <0.9. Further provided are methods wherein the identifying the sample as having atopic dermatitis is based a pathology score >1.0. Further provided are methods wherein the identifying the sample as having psoriasis is based a pathology score <1.0. Further provided are embodiments wherein the subject is in remission or has undergone treatment and has low to no symptoms. Further provided are embodiments wherein the subject is presently experiencing an inflammatory response or reaction. Further provided are embodiments wherein the subject is not presently experiencing an inflammatory response or reaction. Further provided are methods wherein the sample is obtained from a subject suspected as having moderate to severe atopic dermatitis or moderate to severe psoriasis. Further provided are methods wherein the pathology score further identifies a disease subtype and/or a disease endotype. Further provided are methods wherein the method further comprises administering a treatment specific to atopic dermatitis or psoriasis based on the identification of the sample as having atopic dermatitis or psoriasis and/or the pathology score. Further provided are methods wherein the subject is a human subject.

Provided herein are systems for differentiating inflammatory skin diseases comprising: a sample device configured to extract a sample from a subject; a sample processing device configured to extract nucleic acids and measure one or more biomarkers associated with the nucleic acids; at least one computer processor configured to (1) receive data comprising the one or more biomarkers and (2) execute an algorithm to process the data and output a pathology score; and a communication module for transmission of the pathology score or a visual interface configured to display the pathology score. Further provided are systems wherein the algorithm is configured to generate the pathology score from a ratio of one or more gene expression levels. Further provided are systems wherein the algorithm is configured to generate the pathology score by: adding expression levels of a first target gene to generate a first value; adding expression levels of a second target gene to generate a second value; and dividing the first value by the second value to generate the pathology score. Further provided are systems, wherein increased expression of the first target gene is associated with atopic dermatitis. Further provided are systems wherein increased expression of the second target gene is associated with psoriasis. Further provided are systems wherein the pathology score greater than 1 is indicative of atopic dermatitis in the sample. Further provided are systems wherein the pathology score less than 1 is indicative of psoriasis in the sample. Further provided are systems wherein the first target gene is independently selected from the group consisting of IL-4R, IL-13, CCL17/TARC, and CCL26/Eotaxin 3. Further provided are systems wherein the second target gene is independently selected from the group consisting of IL-17A, IL-22, IL-23A, NOS2, S100A8, S100A9, CXCL9, and CXCL10. Further provided are systems wherein the first target gene is IL-13 or CCL17/TARC. Further provided are systems wherein second target gene is IL-17A or NOS2 Further provided are systems wherein the algorithm is configured to generate the pathology score by: adding expression levels of a first and a second target gene to generate a first value; adding expression levels of a third and a fourth target gene to generate a second value; and dividing the first value by the second value to generate the pathology score. Further provided are systems, wherein increased expression of the first target gene and/or the second target gene are associated with atopic dermatitis. Further provided are systems wherein increased expression of the third target gene and/or the fourth target gene are associated with psoriasis. Further provided are systems wherein the pathology score greater than 1 is indicative of atopic dermatitis in the sample. Further provided are systems wherein the pathology score less than 1 is indicative of psoriasis in the sample. Further provided are systems wherein the first target gene and the second target gene are independently selected from the group consisting of IL-4R, IL-13, CCL17/TARC, and CCL26/Eotaxin 3. Further provided are systems wherein the third target gene and the fourth target gene are independently selected from the group consisting of IL-17A, IL-22, IL-23A, NOS2, S100A8, S100A9, CXCL9, and CXCL10. Further provided are systems wherein the first target gene and the second target gene are IL-13 and CCL17/TARC, respectively. Further provided are systems wherein third target gene and the fourth target gene are IL-17A and NOS2, respectively. Further provided are systems wherein the sample processing device comprises a DNA sequencer, a qPCR instrument, and/or a mass array instrument. Further provided are systems wherein the subject is a human subject.

Provided herein are compositions comprising: one or more target nucleic acid molecules derived from a non-invasive sample obtained from a subject; and one or more probes configured to bind to the one or more target nucleic acid molecules, wherein the one or more target nucleic acid molecules correspond to one or more target genes, and wherein the one or more target genes is differentially expressed in atopic dermatitis vs. psoriasis. Further provided are compositions wherein the target nucleic acid molecules comprises DNA and/or RNA. Further provided are compositions wherein the DNA target nucleic acid molecule is a cDNA molecule. Further provided are compositions wherein at least one of the one or more probes is hybridized to the one or more target nucleic acid molecules. Further provided are compositions wherein the one or more target nucleic acid molecules corresponds to one or more target gene selected from the group consisting of IL-4R, IL-13, CCL17/TARC, CCL26/Eotaxin 3, IL-17A, IL-22, IL-23A, NOS2, S100A8, S100A9, CXCL9, and CXCL10. Further provided are compositions wherein the one or more target nucleic acid molecules corresponds to one or more target gene selected from the group consisting of IL-13, CCL17/TARC, NOS2, and IL-17A. Further provided are compositions wherein the one or more probes comprises a reporter moiety. Further provided are compositions wherein the reporter moiety comprises a fluorescent label or a mass label. Further provided are compositions wherein the composition further comprises at least one primer specific to the one or more target nucleic acid molecules. Further provided are compositions wherein the composition further comprises at least one polymerase. Further provided are compositions wherein the subject is a human subject. Further provided are embodiments wherein the subject is in remission or has undergone treatment and has low to no symptoms. Further provided are embodiments wherein the subject is presently experiencing an inflammatory response or reaction. Further provided are embodiments wherein the subject is not presently experiencing an inflammatory response or reaction. Further provided are compositions wherein the human subject is suspected of having an inflammatory disease. Further provided are compositions wherein the inflammatory disease is atopic dermatitis or psoriasis. Further provided are compositions wherein the atopic dermatitis or the psoriasis is moderate to severe. Further provided are compositions wherein the inflammatory disease is characterized by abnormalities in the skin barrier and/or chronic S. aureus colonization. Further provided are compositions, wherein the sample is an epidermal skin sample. Further provided are compositions wherein the sample is a lesional skin sample. Further provided are compositions wherein the sample is a non-lesional skin sample. Further provided are compositions wherein the non-invasive sample is collected using one or more adhesive patches. Further provided are compositions wherein the one or more adhesive patches is a DermTech SmartSticker® or a D-SQAME® skin stripping disc. Further provided are compositions wherein the differential expression of the one or more target genes provides a molecular signature indicative of atopic dermatitis or psoriasis.

Provided herein are compositions comprising: a buffer; an adhesive patch or portion of an adhesive patch; and an epidermal skin sample comprising one or more target molecules. Further provided are compositions wherein the one or more target molecules is selected from the group consisting of a protein, RNA, DNA, and lipid. Further provided are compositions wherein the epidermal skin sample comprises at least 1.5 mg of stratum corneum tissue. Further provided are compositions wherein the epidermal skin sample is a lesional skin sample. Further provided are compositions wherein the epidermal skin sample is a non-lesional skin sample. Further provided are compositions wherein the composition does not comprise a fixative reagent. Further provided are compositions wherein the composition is stable for up to 10 days. Further provided are compositions wherein the epidermal skin sample comprises one or more cell types selected from the group consisting of keratinocytes, T-cells, dendritic cells, and melanocytes. Further provided are compositions wherein the one or more target molecules is a target nucleic acid molecule. Further provided are compositions wherein the target nucleic acid molecule is an RNA molecule. Further provided are compositions wherein the target nucleic acid molecule is a cDNA molecule. Further provided are compositions wherein the RNA or cDNA molecule corresponds to one or more genes selected from the group consisting of IL-4R, IL-13, CCL17/TARC, CCL26/Eotaxin 3, IL-17A, IL-22, IL-23A, NOS2, S100A8, S100A9, CXCL9, and CXCL10. Further provided are compositions wherein the RNA or cDNA molecule corresponds to one or more genes selected from the group consisting of IL-13, CCL17/TARC, IL-17A, and NOS2. Further provided are compositions wherein the epidermal skin sample is from a region of the skin exhibiting histopathology indicative of epithelial acanthosis and/or mononuclear perivascular infiltrate. Further provided are compositions wherein the epidermal skin sample is from a region of the skin not exhibiting histopathology Further provided are compositions wherein the target molecule is associated with a microbiome. Further provided are compositions wherein the epidermal skin sample is obtained from a human subject. Further provided are compositions wherein the human subject is suspected of having an inflammatory disease. Further provided are compositions wherein the inflammatory disease is atopic dermatitis or psoriasis.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows normalized gene expression levels for various genes with confidence intervals indicating which expression levels are associated with atopic dermatitis (left set of bars) or psoriasis (right set of bars). The y-axis is labeled normalized expression from 0 to 25 at 5 unit intervals.

FIG. 2A shows a ratio of normalized expression levels of four genes determined by a machine learning model. The x-axis is labeled 1-specificity from 0.0 to 1.0 at 0.2 unit intervals; the y-axis is labeled sensitivity from 0.0 to 1.0 at 0.2 unit intervals. The depicted ratio is (CCL17+IL13)/(IL17A+NOS2), and the AUC is 0.9525.

FIG. 2B depicts separation of disease classes using the IL17A, NOS2, CCL17, and IL13 genes grouped by principal competent analysis. The x-axis is labeled Dimension 1 (53.1%) from −2 to 2 at 1 unit intervals; the y-axis is labeled Dimension 2 (24.7%) from −1 to 3 at 1 unit intervals. Triangles represent lesional psoriasis samples and circles represent lesional atopic dermatitis samples.

FIG. 3 shows example data using a ratio calculated by a machine learning model and the accuracy of machine learning model predictions. Atopic dermatitis: % QNS: 28% (14 out of 50); % Match b/w Clinical Diagnosis and Molecular Results: 88.9% (32 out of 36). Psoriasis: % QNS: 8%

### (4 out of 50) % Match b/w Clinical Diagnosis and Molecular Results: 76.1% (35 out of 46).

FIG. 4A shows example ratio data for the 36 subjects diagnosed with atopic dermatitis (AD) and the 46 subjects diagnosed with psoriasis (PS). The y-axis is labeled from 0.0009765625 to 1024 on a base 32 logarithmic scale.

FIG. 4B shows a revised ratio determined by the machine learning model after receiving feedback regarding the 36 subjects diagnosed with atopic dermatitis and the 46 subjects diagnosed with psoriasis. The x-axis is labeled 100%—specificity from 0 to 100 at 20 unit intervals; the y-axis is labeled sensitivity % from 0 to 100 at 20 unit intervals.

FIG. 5 shows an example system for diagnosing atopic dermatitis and/or psoriasis.

FIG. 6 shows an example machine learning process for providing an indication of atopic dermatitis and/or psoriasis.

FIG. 7 shows an example machine learning process for providing an indication of atopic dermatitis or psoriasis.

FIG. 8 shows an example system for training a machine learning model.

FIG. 9 depicts a workflow for determining if a subject has atopic dermatitis or psoriasis.

FIG. 10 depicts a computer system that is programmed or otherwise configured to implement methods provided herein.

FIG. 11 depicts one or more example processing devices in communication with one or more other processing devices.

FIG. 12 depicts one or more example processing devices in communication with a network.

### DETAILED DESCRIPTION OF THE DISCLOSURE

Autoimmune skin disorders occur when a person's own immune systems mistakenly attacks healthy cells. Exemplary skin disorders comprise, but are not limited to, psoriasis, lupus, and atopic dermatitis.

Psoriasis is a persistent and chronic skin condition that can change the life cycle of skin cells. Psoriasis can cause cells to build up rapidly on the surface of the skin. The extra skin cells can form thick, silvery scales and itchy, dry, red patches that are sometimes painful.

Atopic dermatitis is a chronic disease that affects the skin. In atopic dermatitis, the skin becomes extremely itchy. Scratching leads to redness, swelling, cracking, “weeping” clear fluid, and finally, crusting and scaling. In most cases, there are periods of exacerbations followed by periods of remissions. Although it is difficult to identify exactly how many people are affected by atopic dermatitis, an estimated 20% of infants and young children experience symptoms of the disease. Approximately 60% of these infants continue to have one or more symptoms of atopic dermatitis in adulthood. Thus, more than 15 million people in the United States have symptoms of the disease. The “lesion area” is the region of the skin affected by atopic dermatitis. Generally a lesion is characterized by skin dryness (xerosis), redness, blisters, scabs, or any combination. A non-lesion area is not affected by atopic dermatitis or any other skin pathology.

With similar symptoms in some cases, differentiating atopic dermatitis and psoriasis may not be a simple process. In particular, when the symptoms are similar, there exists a chance for misdiagnosis, by diagnosing one condition rather than a condition that a subject actually has. Accordingly, there exists a need in the art to reliably differentiate atopic dermatitis and psoriasis quickly, efficiently, and accurately.

Described herein are systems and methods for differentiating atopic dermatitis and psoriasis using at least a ratio of expression levels of relevant target genes. In particular, genes such as IL13, IL4R, CCL17, CCL26, IL23A, IL22, S100A7, S100A8, S100A9, CXCL9, CXCL10, CXCL11, NOS2, IL17A, IL13R, CCL19, CCL27, TSLP, CCL18, IL23A, IL4R, and IL31 may be expressed differently in samples with atopic dermatitis from samples with psoriasis. By utilizing those differences in expression, a ratio between certain expression levels may be determined, indicating atopic dermatitis or psoriasis (or vice versa) as the ratio increase or decreases. Thus, retrieving a skin sample and determining those expression levels, diagnosis of atopic dermatitis and psoriasis can be done both more efficiently and accurately. Even further, through use of software incorporating the use of a machine learning model, the results can be delivered even more efficiently and feedback can be provided to the machine learning model, allowing for an even more accurate ratio to be determined with each skin sample received.

Thus, by utilizing the gene expression levels in order to create a ratio, patients may be more readily, quickly, and accurately diagnosed as having atopic dermatitis or psoriasis.

### Psoriasis Gene Classifiers and Methods of Use

In some embodiments, disclosed herein is a method of detecting the expression level of a gene from a gene classifier, which is associated with psoriasis. In some instances, the method comprises detecting the expression level of Interleukin 17A (IL-17A), Interleukin 22 (IL-22), Interleukin 23 (IL-23), S100 Calcium Binding Protein A8 (S100A8), S100 Calcium Binding Protein A9 (S100A9), (NOS2), or a combination thereof. In some instances, the method comprises (a) isolating nucleic acids from a skin sample obtained from the subject, wherein the skin sample (e.g., comprising cells from the stratum corneum); and (b) detecting the expression level of IL-17A, IL-22, IL-23, S100A8, S100A9, NOS2, or a combination thereof, by contacting the isolated nucleic acids with a set of probes that recognizes IL-17A, IL-22, IL-23, S100A8, S100A9, NOS2, or a combination thereof, and detects binding between IL-17A, IL-22, IL-23, S100A8, S100A9, NOS2, or a combination thereof and the set of probes.

In some embodiments, the method comprises detecting the expression levels of two or more, three or more, four or more, or five or more of genes from the gene classifier: IL-17A, IL-22, IL-23, S100A8, S100A9, and NOS2. In some cases, the method comprises detecting the expression levels of IL-17A, IL-22, IL-23, S100A8, S100A9, and NOS2. In some cases, the method comprises detecting the expression levels of IL-17A, IL-22, IL-23, S100A8, S100A9, and NOS2. In some cases, the method comprises detecting the expression levels of IL-17A, IL-22, IL-23, S100A8, S100A9, and NOS2. In some cases, the method comprises detecting the expression levels of IL-17A, IL-22, IL-23, S100A8, S100A9, and NOS2. In some cases, the method comprises detecting the expression levels of IL-17A, IL-22, IL-23, S100A8, S100A9, and NOS2. In some cases, the method comprises detecting the expression levels of one or more of IL-13, IL-4R, CCL17, CCL27, IL-23A, IL-22, S100A7, CXCL9, CXCL10, CXCL11, IL-13R, CCL10, CCL27, TSLP, CCL18, IL-17A, IL-22, IL-23, S100A8, S100A9, and NOS2.

In some instances, the expression level is an upregulated gene expression level. In some instances, the expression level is an upregulated gene expression level, compared to a gene expression level of an equivalent gene from a control sample. In some cases, the control sample is a normal skin sample. In some cases, the gene expression level of IL-17A, IL-22, IL-23, S100A8, S100A9, NOS2, or a combination thereof is upregulated. In some instances, the upregulated gene expression level occurs in areas of skin comprising psoriatic plaques.

In some instances, the gene expression level of IL-17A, IL-22, IL-23, S100A8, S100A9, and/or NOS2 is increased by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some cases, the gene expression level of IL-17A, IL-22, IL-23, S100A8, S100A9, and/or NOS2 is increased by at least 10-fold. In some cases, the gene expression level of IL-17A, IL-22, IL-23, S100A8, S100A9, and/or NOS2 is increased by at least 20-fold. In some cases, the gene expression level of IL-17A, IL-22, IL-23, S100A8, S100A9, and/or NOS2 is increased by at least 30-fold. In some cases, the gene expression level of IL-17A, IL-22, IL-23, S100A8, S100A9, and/or NOS2 is increased by at least 40-fold. In some cases, the gene expression level of IL-17A, IL-22, IL-23, S100A8, S100A9, and/or NOS2 is increased by at least 50-fold. In some cases, the gene expression level of IL-17A, IL-22, IL-23, S100A8, S100A9, and/or NOS2 is increased by at least 80-fold. In some cases, the gene expression level of IL-17A, IL-22, IL-23, S100A8, S100A9, and/or NOS2 is increased by at least 100-fold. In some cases, the gene expression level of IL-17A, IL-22, IL-23, S100A8, S100A9, and/or NOS2 is increased by at least 130-fold. In some cases, the gene expression level of IL-17A, IL-22, IL-23, S100A8, S100A9, and/or NOS2 is increased by at least 150-fold. In some cases, the gene expression level of IL-17A, IL-22, IL-23, S100A8, S100A9, and/or NOS2 is increased by at least 200-fold. In some cases, the gene expression level of IL-17A, IL-22, IL-23, S100A8, S100A9, and/or NOS2 is increased by at least 300-fold. In some cases, the gene expression level of IL-17A, IL-22, IL-23, S100A8, S100A9, and/or NOS2 is increased by at least 500-fold. In some cases, the increased gene expression level is compared to a gene expression level of an equivalent gene from a control sample. In some cases, the control sample is a normal skin sample. In some cases, the control sample is non-lesional or asymptomatic skin sample from the same subject that a test sample is obtained. In some cases, the control sample is non-lesional or an asymptomatic skin sample from a different subject. In some cases, a control sample is not obtained. In some instances, the up-regulated gene expression level occurs in areas of skin comprising psoriatic plaques.

In some cases, the gene expression level of IL-17A, IL-22, IL-23, S100A8, S100A9, and/or NOS2 is increased by at least 10%, 20%, 30%, 40%, 50%, 60%, 70%, 80%, 90%, 100%, 200%, 300%, 400%, 500%, or more. In some cases, the gene expression level of IL-17A, IL-22, IL-23, S100A8, S100A9, and/or NOS2 is increased by at least 10%. In some cases, the gene expression level of IL-17A, IL-22, IL-23, S100A8, S100A9, and/or NOS2 is increased by at least 20%. In some cases, the gene expression level of IL-17A, IL-22, IL-23, S100A8, S100A9, and/or NOS2 is increased by at least 30%. In some cases, the gene expression level of IL-17A, IL-22, IL-23, S100A8, S100A9, and/or NOS2 is increased by at least 40%. In some cases, the gene expression level of IL-17A, IL-22, IL-23, S100A8, S100A9, and/or NOS2 is increased by at least 50%. In some cases, the gene expression level of IL-17A, IL-22, IL-23, S100A8, S100A9, and/or NOS2 is increased by at least 80%. In some cases, the gene expression level of IL-17A, IL-22, IL-23, S100A8, S100A9, and/or NOS2 is increased by at least 90%. In some cases, the gene expression level of IL-17A, IL-22, IL-23, S100A8, S100A9, and/or NOS2 is increased by at least 100%. In some cases, the gene expression level of IL-17A, IL-22, IL-23, S100A8, S100A9, and/or NOS2 is increased by at least 150%. In some cases, the gene expression level of IL-17A, IL-22, IL-23, S100A8, S100A9, and/or NOS2 is increased by at least 200%. In some cases, the gene expression level of IL-17A, IL-22, IL-23, S100A8, S100A9, and/or NOS2 is increased by at least 300%. In some cases, the gene expression level of IL-17A, IL-22, IL-23, S100A8, S100A9, and/or NOS2 is increased by at least 500%. In some cases, the increased gene expression level is compared to a gene expression level of an equivalent gene from a control sample. In some cases, the control sample is a normal skin sample. In some instances, the down-regulated gene expression level occurs in areas of skin comprising psoriatic plaques.

In some embodiments, the set of probes recognizes at least one but no more than six genes selected from IL-17A, IL-22, IL-23, S100A8, S100A9, and NOS2. In some cases, the set of probes recognizes IL-17A, and/or IL-22. In some cases, the set of probes recognizes IL-17A and IL-23. In some cases, the set of probes recognizes IL-17F and/or S100A8. In some cases, the set of probes recognizes IL-17F and/or S100A9. In some cases, the set of probes recognizes IL-17A and/or NOS2. In some cases, the set of probes recognizes IL-17A, IL-22, IL-23, S100A8, S100A9, and/or NOS2.

In some embodiments, the method further comprises detecting the expression levels of Interleukin 17C (IL-17C), S100 Calcium Binding Protein A7 (S100A7), Interleukin 17 Receptor A (IL-17RA), Interleukin 17 Receptor C (IL-17RC), Interleukin 23 Subunit Alpha (IL-23A), Interleukin 22 (IL-22), Interleukin 26 (IL-26), Interleukin 24 (IL-24), Interleukin 6 (IL-6), C-X-C Motif Chemokine Ligand 1 (CXCL1), Interferon Gamma (IFN-gamma), Interleukin 31, (IL-31), Interleukin 33 (IL-33), Tumor Necrosis Factor (TNFα), Lipocalin 2 (LCN2), C-C Motif Chemokine Ligand 20 (CCL20), TNF Receptor Superfamily Member 1A (TNFRSF1A) or a combination thereof. In some cases, the detecting comprises contacting the isolated nucleic acids with an additional set of probes that recognizes IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, TNFRSF1A, or a combination thereof, and detects binding between IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, TNFRSF1A, or a combination thereof and the additional set of probes.

In some cases, the additional set of probes recognizes one but no more than ten genes. In some cases, the additional set of probes recognizes 2, 3, 4, 5, 6, 7, 8, 9, 10, 11, 12, 13, 14, 15, 16 or 17 genes selected from IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, and TNERSF1A.

In some cases, the expression level of one or more genes selected from IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, and TNERSF1A is an elevated gene expression level. In such cases, the gene expression level is elevated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some instances, the gene expression level is elevated by at least 10%, 20%, 30%, 40%, 50%, 60%, 70%, 80%, 90%, 100%, 200%, 300%, 400%, 500%, or more. In some instances, the expression level is compared to a gene expression level of an equivalent gene from a control sample. In some instances, the control sample is a normal skin sample.

In some embodiments, a method described herein further comprises detecting a skin region affected with psoriasis. In some cases, also described herein include a method monitoring the skin region affected with psoriasis, for about 1 week, 2 weeks, 3 weeks, 1 month, 2 months, 6 months, or more.

In some instances, the method has an improved specificity over conventional methods, of at least or about 70%, 75%, 80%, 85%, 90%, or more than 95% when detecting the gene expression level of IL-17A, IL-17F, IL-8, CXCL5, S100A9, DEFB, or a combination thereof. In some embodiments, the specificity is at least or about 70%, 75%, 80%, 85%, 90%, or more than 95% when detecting the gene expression level of IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, TNFRSF1A, or a combination thereof.

In some cases, the method also has an improved sensitivity over conventional methods. In some embodiments, the sensitivity is at least or about 70%, 75%, 80%, 85%, 90%, or more than 95% when detecting the gene expression levels of IL-17A, IL-17F, IL-8, CXCL5, S100A9, DEFB, or a combination thereof over conventional methods. In some cases, the sensitivity is at least or about 70%, 75%, 80%, 85%, 90%, or more than 95% when detecting the gene expression levels of IL-17C, S100A7, IL-17RA, IL-17RC, IL-23A, IL-22, IL-26, IL-24, IL-6, CXCL1, IFN-gamma, IL-31, IL-33, TNFα, LCN2, CCL20, TNFRSF1A, or a combination thereof.

In some embodiments, a method described herein comprises detecting gene expression levels from a first gene classifier and a second gene classifier in a subject in need thereof, comprising: (a) isolating nucleic acids from a skin sample obtained from the subject, wherein the skin sample (e.g., comprising cells from the stratum corneum); (b) detecting the expression levels of one or more genes from the first gene classifier: IL-17A, IL-17F, IL-8, CXCL5, S100A9, and DEFB, by contacting the isolated nucleic acids with a set of probes that recognizes one or more genes from the first gene classifier, and detects binding between one or more genes from the first gene classifier and the set of probes; and (c) detecting the expression levels of one or more genes from the second gene classifier: IL-17C, S100A7, IL-17RA, IL-17RC, IL-23A, IL-22, IL-6, IL-24, IL-6, CXCL1, IFN-gamma, IL-31, IL-33, TNFα, LCN2, CCL20, and TNERSF1A, by contacting the isolated nucleic acids with an additional set of probes that recognizes one or more genes from the second gene classifier, and detects binding between one or more genes from the second gene classifier and the additional set of probes.

In some embodiments, also provided herein is a method of treating psoriasis, which comprises administering one or more inhibitors. Inhibitors for inclusion in methods for treatment of psoriasis described herein include, but are not limited to, inhibitors of TNFα, IL-17A, and IL-23. Exemplary inhibitors of TNFα include, but are not limited to, adalimumab, certolizumab, etanercept, golimumab, and infliximab. Exemplary inhibitors of IL-17A include, but are not limited to, ixekizumab (LY2439821), brodalumab (AMG 827), and secukinumab. Exemplary inhibitors of IL-23 include, but are not limited to, guselkumab, tildrakizumab, and risankizumab.

In some cases, the inhibitor for inclusion in the methods described herein for treatment of psoriasis is an inhibitor of TNFα. In some cases, the subject is treated with an inhibitor of TNFα such as adalimumab, certolizumab, etanercept, golimumab, or infliximab.

In some cases, the inhibitor for inclusion in the methods described herein for treatment of psoriasis is an inhibitor of IL-17A. In some cases, the subject is treated with an inhibitor of IL-17A such as ixekizumab (LY2439821), brodalumab (AMG 827), or secukinumab.

In some cases, the inhibitor for inclusion in the methods described herein for treatment of psoriasis is an inhibitor of IL-23. In some cases, the subject is treated with an inhibitor of IL-23 such as guselkumab, tildrakizumab, or risankizumab.

### Atopic Dermatitis Gene Classifiers and Methods of Use

In some embodiments, disclosed herein is a method of detecting the expression level of a gene from a gene classifier, which is associated with atopic dermatitis. In some instances, the method comprises detecting the expression level of Interleukin 13 (IL-13), Interleukin 31 (IL-31), Thymic Stromal Lymphopoietin (TSLP), or a combination thereof. In some instances, the method comprises (a) isolating nucleic acids from a skin sample obtained from the subject, wherein the skin sample (e.g., comprising cells from the stratum corneum); and (b) detecting the expression level of IL-13, IL-31, TSLP, or a combination thereof, by contacting the isolated nucleic acids with a set of probes that recognizes IL-13, IL-31, TSLP, or a combination thereof, and detects binding between IL-13, IL-31, TSLP, or a combination thereof and the set of probes.

In some embodiments, the method comprises detecting the expression levels of two or more, or three or more of genes from the gene classifier: IL-13, IL-31, and TSLP. In some cases, the method comprises detecting the expression levels of IL-13, IL-31, and TSLP. In some cases, the method comprises detecting the expression levels of IL-31 and TSLP. In some cases, the method comprises detecting the expression levels of IL-13 and IL-31. In some cases, the method comprises detecting the expression levels of IL-13 and TSLP.

In some instances, the expression level is an upregulated gene expression level. In some instances, the expression level is an up-regulated gene expression level, compared to a gene expression level of an equivalent gene from a control sample. In some cases, the control sample is a normal skin sample. In some cases, the gene expression level of IL-13, IL-31, TSLP, or a combination thereof is up-regulated. In some instances, the up-regulated gene expression level occurs in areas of skin comprising atopic dermatitis.

In some instances, the gene expression level of IL-13, IL-31, or TSLP is increased by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some cases, the gene expression level of IL-13, IL-31, or TSLP is increased by at least 10-fold. In some cases, the gene expression level of IL-13, IL-31, or TSLP is increased by at least 20-fold. In some cases, the gene expression level of IL-13, IL-31, or TSLP is increased by at least 30-fold. In some cases, the gene expression level of IL-13, IL-31, or TSLP is increased by at least 40-fold. In some cases, the gene expression level of IL-13, IL-31, or TSLP is increased by at least 50-fold. In some cases, the gene expression level of IL-13, IL-31, or TSLP is increased by at least 80-fold. In some cases, the gene expression level of IL-13, IL-31, or TSLP is increased by at least 100-fold. In some cases, the gene expression level of IL-13, IL-31, or TSLP is increased by at least 130-fold. In some cases, the gene expression level of IL-13, IL-31, or TSLP is increased by at least 150-fold. In some cases, the gene expression level of IL-13, IL-31, or TSLP is increased by at least 200-fold. In some cases, the gene expression level of IL-13, IL-31, or TSLP is increased by at least 300-fold. In some cases, the gene expression level of IL-13, IL-31, or TSLP is increased by at least 500-fold. In some cases, the decreased gene expression level is compared to a gene expression level of an equivalent gene from a control sample. In some cases, the control sample is a normal skin sample. In some instances, the down-regulated gene expression level occurs in areas of skin comprising atopic dermatitis.

In some cases, the gene expression level of IL-13, IL-31, or TSLP is increased by at least 10%, 20%, 30%, 40%, 50%, 60%, 70%, 80%, 90%, 100%, 200%, 300%, 400%, 500%, or more. In some cases, the gene expression level of IL-13, IL-31, or TSLP is increased by at least 10%. In some cases, the gene expression level of IL-13, IL-31, or TSLP is increased by at least 20%. In some cases, the gene expression level of IL-13, IL-31, or TSLP is increased by at least 30%. In some cases, the gene expression level of IL-13, IL-31, or TSLP is increased by at least 40%. In some cases, the gene expression level of IL-13, IL-31, or TSLP is increased by at least 50%. In some cases, the gene expression level of IL-13, IL-31, or TSLP is increased by at least 80%. In some cases, the gene expression level of IL-13, IL-31, or TSLP is increased by at least 90%. In some cases, the gene expression level of IL-13, IL-31, or TSLP is increased by at least 100%. In some cases, the gene expression level of IL-13, IL-31, or TSLP is increased by at least 150%. In some cases, the gene expression level of IL-13, IL-31, or TSLP is increased by at least 200%. In some cases, the gene expression level of IL-13, IL-31, or TSLP is increased by at least 300%. In some cases, the gene expression level of IL-13, IL-31, or TSLP is increased by at least 500%. In some cases, the decreased gene expression level is compared to a gene expression level of an equivalent gene from a control sample. In some cases, the control sample is a normal skin sample. In some instances, the down-regulated gene expression level occurs in areas of skin comprising atopic dermatitis.

In some embodiments, the set of probes recognizes at least one but no more than three genes selected from IL-13, IL-31, and TSLP. In some cases, the set of probes recognizes IL-13 and IL-31. In some cases, the set of probes recognizes IL-31 and TSLP. In some cases, the set of probes recognizes IL-13 and TSLP. In some cases, the set of probes recognizes IL-13, IL-31, and TSLP.

In some embodiments, the method further comprises detecting the expression levels of Interleukin 13 Receptor (IL-13R), Interleukin 4 Receptor (IL-4R), Interleukin 17 (IL-17), Interleukin 22 (IL-22), C-X-C Motif Chemokine Ligand 9 (CXCL9), C-X-C Motif Chemokine Ligand 10 (CXCL10), C-X-C Motif Chemokine Ligand 10 (CXCL11), S100 Calcium Binding Protein A7 (S100A7), S100 Calcium Binding Protein A8 (S100A8), S100 Calcium Binding Protein A9 (S100A9), C-C Motif Chemokine Ligand 17 (CCL17), C-C Motif Chemokine Ligand 18 (CCL18), C-C Motif Chemokine Ligand 19 (CCL19), C-C Motif Chemokine Ligand 26 (CCL26), C-C Motif Chemokine Ligand 27 (CCL27), Nitric Oxide Synthetase 2 (NOS2) or a combination thereof. In some cases, the detecting comprises contacting the isolated nucleic acids with an additional set of probes that recognizes IL-13R, IL-4R, IL-17, IL-22, CXCL9, CXCL10, CXCL11, S100A7, S100A8, S100A9, CCL17, CCL18, CCL19, CCL26, CCL27, NOS2, or a combination thereof, and detects binding between IL-13R, IL-4R, IL-17, IL-22, CXCL9, CXCL10, CXCL11, S100A7, S100A8, S100A9, CCL17, CCL18, CCL19, CCL26, CCL27, NOS2, or a combination thereof and the additional set of probes.

In some cases, the additional set of probes recognizes one but no more than ten genes. In some cases, the additional set of probes recognizes 2, 3, 4, 5, 6, 7, 8, 9, 10, 11, 12, 13, 14, 15, or 16 genes selected from IL-13R, IL-4R, IL-17, IL-22, CXCL9, CXCL10, CXCL11, S100A7, S100A8, S100A9, CCL17, CCL18, CCL19, CCL26, CCL27, and NOS2.

In some cases, the expression level of one or more genes selected from IL-13R, IL-4R, IL-17, IL-22, CXCL9, CXCL10, CXCL11, S100A7, S100A8, S100A9, CCL17, CCL18, CCL19, CCL26, CCL27, and NOS2 is an elevated gene expression level. In such cases, the gene expression level is elevated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some instances, the gene expression level is elevated by at least 10%, 20%, 30%, 40%, 50%, 60%, 70%, 80%, 90%, 100%, 200%, 300%, 400%, 500%, or more. In some instances, the expression level is compared to a gene expression level of an equivalent gene from a control sample. In some instances, the control sample is a normal skin sample.

In some embodiments, a method described herein further comprises detecting a skin region affected with atopic dermatitis. In some cases, also described herein include a method monitoring the skin region affected with atopic dermatitis, for about 1 week, 2 weeks, 3 weeks, 1 month, 2 months, 6 months, or more.

In some instances, the method has an improved specificity, of at least or about 70%, 75%, 80%, 85%, 90%, or more than 95% when detecting the gene expression level of IL-13, IL-31, TSLP, or a combination thereof. In some embodiments, the specificity is at least or about 70%, 75%, 80%, 85%, 90%, or more than 95% when detecting the gene expression level of IL-13R, IL-4R, IL-17, IL-22, CXCL9, CXCL10, CXCL11, S100A7, S100A8, S100A9, CCL17, CCL18, CCL19, CCL26, CCL27, NOS2, or a combination thereof.

In some cases, the method also has an improved sensitivity. In some embodiments, the sensitivity is at least or about 70%, 75%, 80%, 85%, 90%, or more than 95% when detecting the gene expression levels of IL-13, IL-31, TSLP, or a combination thereof. In some cases, the sensitivity is at least or about 70%, 75%, 80%, 85%, 90%, or more than 95% when detecting the gene expression levels of IL-13R, IL-4R, IL-17, IL-22, CXCL9, CXCL10, CXCL11, S100A7, S100A8, S100A9, CCL17, CCL18, CCL19, CCL26, CCL27, NOS2, or a combination thereof.

In some embodiments, a method described herein comprises detecting gene expression levels from a first gene classifier and a second gene classifier in a subject in need thereof, comprising: (a) isolating nucleic acids from a skin sample obtained from the subject, wherein the skin sample (e.g., comprising cells from the stratum corneum); (b) detecting the expression levels of one or more genes from the first gene classifier: IL-13, IL-31, and TSLP, by contacting the isolated nucleic acids with a set of probes that recognizes one or more genes from the first gene classifier, and detects binding between one or more genes from the first gene classifier and the set of probes; and (c) detecting the expression levels of one or more genes from the second gene classifier: IL-13R, IL-4R, IL-17, IL-22, CXCL9, CXCL10, CXCL11, S100A7, S100A8, S100A9, CCL17, CCL18, CCL19, CCL26, CCL27, and NOS2, by contacting the isolated nucleic acids with an additional set of probes that recognizes one or more genes from the second gene classifier, and detects binding between one or more genes from the second gene classifier and the additional set of probes.

Provided herein are methods for treatment of atopic dermatitis comprising administering one or more inhibitors described herein. Inhibitors for inclusion in methods for treatment of atopic dermatitis described herein include, but are not limited to, inhibitors of IL-13, PDE4, or IL-31. In some cases, the inhibitor for inclusion in the methods described herein for treatment of atopic dermatitis is an inhibitor of IL-13. In some cases, the inhibitor for inclusion in the methods described herein for treatment of atopic dermatitis is an inhibitor of PDE4. In some cases, the inhibitor for inclusion in the methods described herein for treatment of atopic dermatitis is an inhibitor of IL-31.

In some cases, the inhibitor of IL-13 includes, but is not limited to, lebrikizumab and tralokinumab. In some cases, the inhibitor of IL-13 is lebrikizumab. In some cases, the inhibitor of IL-13 is tralokinumab. In some cases, a subject is treated with an inhibitor of IL-13 such as lebrikizumab or tralokinumab.

In some instances, the PDE4 inhibitor includes, but is not limited to, crisaborole. In some instances, a subject is treated with a PDE4 inhibitor such as crisaborole.

In some instances, the IL-31 inhibitor includes, but is not limited to, nemolizumab. In some instances, a subject is treated with an IL-31 inhibitor such as nemolizumab.

### Diagnostic Tools and Methods

In some embodiments, one or more genes are detected with a set of probes. In some embodiments, the set of probes comprises at least or about 1, 2, 3, 5, 6, 7, 8, 9, 10, 11, 12, 13, 14, 15, 16, 17, 18, 19, 20, 25, 30, or more than 30 probes. In some embodiments, the set of probes comprises about 6 probes. In some embodiments, the set of probes comprises about 7 probes. In some embodiments, the set of probes comprises about 8 probes. In some embodiments, the set of probes comprises about 9 probes. In some embodiments, the set of probes comprises about 10 probes. In some embodiments, the set of probes comprises about 13 probes. In some embodiments, the set of probes comprises about 15 probes. In some embodiments, the set of probes comprises about 20 probes.

In some embodiments, the set of probes comprises one or more primer pairs. In some embodiments, a number of primer pairs is at least or about 1, 2, 3, 5, 6, 7, 8, 9, 10, 11, 12, 13, 14, 15, 16, 17, 18, 19, 20, 25, 30, or more than 30 primer pairs. In some embodiments, the number of primer pairs is about 8 primer pairs. In some embodiments, the number of primer pairs is about 9 primer pairs. In some embodiments, the number of primer pairs is about 10 primer pairs.

In some embodiments, one or more probes in the set of probes is labeled. In some embodiments, the one or more probe is labeled with a radioactive label, a fluorescent label, an enzyme, a chemiluminescent tag, a colorimetric tag, an affinity tag or other labels or tags that are known in the art.

Exemplary affinity tags include, but are not limited to, biotin, desthiobiotin, histidine, polyhistidine, myc, hemagglutinin (HA), FLAG, glutathione S transferase (GST), or derivatives thereof. In some embodiments, the affinity tag is recognized by avidin, streptavidin, nickel, or glutathione.

In some embodiments, the fluorescent label is a fluorophore, a fluorescent protein, a fluorescent peptide, quantum dots, a fluorescent dye, a fluorescent material, or variations or combinations thereof.

Exemplary fluorophores include, but are not limited to, Alexa-Fluor dyes (e.g., Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750), APC, Cascade Blue, Cascade Yellow and R-phycoerythrin (PE), DyLight 405, DyLight 488, DyLight 550, DyLight 650, DyLight 680, DyLight 755, DyLight 800, FITC, Pacific Blue, PerCP, Rhodamine, and Texas Red, Cy5, Cy5.5, Cy7.

Examples of fluorescent peptides include GFP (Green Fluorescent Protein) or derivatives of GFP (e.g., EBFP, EBFP2, Azurite, mKalama1, ECFP, Cerulean, CyPet, YFP, Citrine, Venus, and YPet.

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. In some embodiments, the fluorescein dye is, but not limited to, 5-carboxyfluorescein, fluorescein-5-isothiocyanate, fluorescein-6-isothiocyanate and 6-carboxyfluorescein. In some embodiments, the rhodamine dye is, but not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, and rhodamine 101 sulfonyl chloride (sold under the tradename of TEXAS RED®). In some embodiments, the cyanine dye is Cy3, Cy3B, Cy3.5, Cy5, Cy5.5, Cy7, IRDYE680, Alexa Fluor 750, IRDye800CW, or ICG.

In some embodiments, the gene expression levels of IL-13, TSLP, IL-31, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the gene expression levels of IL-13R, IL-4R, IL-17, IL-22, CXCL9, CXCL10, CXCL11, S100A7, S100A8, S100A9, CCL17, CCL18, CCL19, CCL26, CCL27, NOS2, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the gene expression levels of IL-17A, IL-17F, IL-8, CXCL5, S100A9, DEFB4A, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the gene expression levels of IL-17C, S100A7, IL-17RA, IL-17RC, IL-23A, IL-22, IL-6, IL-24, IL-6, CXCL1, IFN-gamma, IL-31, IL-33, TNFα, LCN2, CCL20, and TNFRSF1A, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the gene expression levels of IFNA1, IFNA2, IFNA4, IFNR1, IFNR2, CCL5, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the gene expression levels of IFNB1, IFNE, IFNW1, ADAR, IFIT, IFI, IRF, OAS1, IRAK1, TNFAIP3, ATG5, TYK2, STAT4, OPN, KRT, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the expression levels are measured using qPCR. In some embodiments, the qPCR comprises use of fluorescent dyes or fluorescent probes. In some embodiments, the fluorescent dye is an intercalating dye. Examples of intercalating dyes include, but are not limited to, intercalating dyes include SYBR green I, SYBR green II, SYBR gold, ethidium bromide, methylene blue, Pyronin Y, DAPI, acridine orange, Blue View, or phycoerythrin. In some embodiments, the qPCR comprises use of more than one fluorescent probe. In some embodiments, the use of more than one fluorescent probes allows for multiplexing. For example, different non-classical variants are hybridized to different fluorescent probes and can be detected in a single qPCR reaction.

### Skin Collection Kit

In some embodiments, the adhesive patch from the sample collection kit described herein comprises a first collection area comprising an adhesive matrix and a second area extending from the periphery of the first collection area. The adhesive matrix is located on a skin facing surface of the first collection area. The second area functions as a tab, suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent.

The matrix material is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive patch comprises a flexible material, enabling the patch to conform to the shape of the skin surface upon application. In some instances, at least the first collection area is flexible. In some instances, the tab is plastic. In an illustrative example, the adhesive patch does not contain latex, silicone, or both. In some embodiments, the adhesive patch is made of a transparent material, so that the skin sampling area of the subject is visible after application of the adhesive patch to the skin surface. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical.

In further embodiments, the adhesive patch of this invention is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector. In some embodiments, one or more adhesive patches is stored after collection. In some embodiments, one or more adhesive patches may be stored in a tube or another collection vesicle, or may be reapplied to a panel or sheet, after collection.

In some instances, nucleic acids are stable on adhesive patch or patches when stored for a period of time or at a particular temperature. In some instances, the period of time is at least or about 1 day, 2 days, 3 days, 4 days, 5 days, 6 days, 7 days, 2 weeks, 3 weeks, 4 weeks, or more than 4 weeks. In some instances, the period of time is about 7 days. In some instances, the period of time is about 10 days. In some instances, the temperature is at least or about −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or more than 50° C. The nucleic acids on the adhesive patch or patches, in some embodiments, are stored for any period of time described herein and any particular temperature described herein. For example, the nucleic acids on the adhesive patch or patches are stored for at least or about 7 days at about 25° C., 7 days at about 30° C., 7 days at about 40° C., 7 days at about 50° C., 7 days at about 60° C., or 7 days at about 70° C. In some instances, the nucleic acids on the adhesive patch or patches are stored for at least or about 10 days at about −80° C.

The peelable release sheet, in certain embodiments, is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the peelable release sheet is configured to hold about 12 adhesive patches. In some instances, the peelable release sheet is configured to hold about 11 adhesive patches. In some instances, the peelable release sheet is configured to hold about 10 adhesive patches. In some instances, the peelable release sheet is configured to hold about 9 adhesive patches. In some instances, the peelable release sheet is configured to hold about 8 adhesive patches. In some instances, the peelable release sheet is configured to hold about 7 adhesive patches. In some instances, the peelable release sheet is configured to hold about 6 adhesive patches. In some instances, the peelable release sheet is configured to hold about 5 adhesive patches. In some instances, the peelable release sheet is configured to hold about 4 adhesive patches. In some instances, the peelable release sheet is configured to hold about 3 adhesive patches. In some instances, the peelable release sheet is configured to hold about 2 adhesive patches. In some instances, the peelable release sheet is configured to hold about 1 adhesive patch.

Provided herein, in certain embodiments, are methods and compositions for obtaining a sample using an adhesive patch, wherein the adhesive patch is applied to the skin and removed from the skin. After removing the used adhesive patch from the skin surface, the patch stripping method, in some instances, further comprise storing the used patch on a placement area sheet, where the patch remains until the skin sample is isolated or otherwise utilized. In some instances, the used patch is configured to be stored on the placement area sheet for at least 1 week at temperatures between −80° C. and 30° C. In some embodiments, the used patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the used patch on the placement area sheet, the removable liner is removed. In some instances, the placement area sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the placement area sheet is configured to hold about 12 adhesive patches. In some instances, the placement area sheet is configured to hold about 11 adhesive patches. In some instances, the placement area sheet is configured to hold about 10 adhesive patches. In some instances, the placement area sheet is configured to hold about 9 adhesive patches. In some instances, the placement area sheet is configured to hold about 8 adhesive patches. In some instances, the placement area sheet is configured to hold about 7 adhesive patches. In some instances, the placement area sheet is configured to hold about 6 adhesive patches. In some instances, the placement area sheet is configured to hold about 5 adhesive patches. In some instances, the placement area sheet is configured to hold about 4 adhesive patches. In some instances, the placement area sheet is configured to hold about 3 adhesive patches. In some instances, the placement area sheet is configured to hold about 2 adhesive patches. In some instances, the placement area sheet is configured to hold about 1 adhesive patch.

The used patch, in some instances, is stored so that the matrix containing, skin facing surface of the used patch is in contact with the placement area sheet. In some instances, the placement area sheet is a panel of the tri-fold skin sample collector. In some instances, the tri-fold skin sample collector further comprises a clear panel. In some instances, the tri-fold skin sample collector is labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. In some instances, the patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances, the placement area panel is a single placement area panel sheet. In some instances, the identity of the skin sample collected is indexed to the tri-fold skin sample collector or placement area panel sheet by using a barcode or printing patient information on the collector or panel sheet. In some instances, the indexed tri-fold skin sample collector or placement sheet is sent to a diagnostic lab for processing. In some instances, the used patch is configured to be stored on the placement panel for at least 1 week at temperatures between −80° C. and 25° C. In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed tri-fold skin sample collector or placement sheet is sent to a diagnostic lab using UPS or FedEx.

In an exemplary embodiment, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The adhesive skin sample collection kit, in some embodiments, comprises at least one adhesive patch, a sample collector, and an instruction for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector, in some instances, further comprises a barcode and/or an area for transcribing patient information. In some instances, the adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provide the kit operator all of the necessary information for carrying out the patch stripping method. The instructions for use sheet preferably include diagrams to illustrate the patch stripping method.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. In some instances, the components of the skin sample collection kit are provided in a cardboard box.

### Cellular Material and Sample Process

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA includes, but not limited to, picogram, nanogram, and microgram quantities.

In some instances, the nucleic acid is a RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (IncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA. DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

Biological samples (e.g., skin samples) for analysis may be obtained using non-invasive techniques, semi-invasive techniques, or minimally invasive techniques. In some instances, a minimally-invasive technique comprises the use of microneedles. In some embodiments, a sample such as a skin sample is collected using one or more microneedles. In some instances, a plurality of microneedles are used to obtain a sample. In some instance, microneedles are polymeric. In some instance, microneedles are coated with a substance (e.g., enzymes, chemical, or other substance) capable of disrupting an extracellular matrix. In some instances, microneedles such as those described in U.S. Pat. No. 10,995,366, incorporated by reference in its entirety, are used to obtain a skin sample. Microneedles in some instances pierce a subject's skin to obtain samples of skin cells, blood, or both. In some instances, microneedles are coated with probes that bind to one or more nucleic acid targets described herein. In some instances, samples are obtained by other methods, such as from FFPE samples or microbiopsies.

Samples may be subjected to one or more assays. In some instances, samples are processed using an automated system. In some instances, the system is configured to accept one or more types of samples. In some instances, the automated system comprises a microfluidics chip. In some instances, a device described in DE102022106693B3 or DE102022111890B3 is used.

Examples of subjects include but are not limited to vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female.

In additional embodiments, the adhered skin sample comprises cellular material including nucleic acids such as RNA or DNA, in an amount that is at least about 1 picogram. In some embodiments, the amount of cellular material is no more than about 1 nanogram. In further or additional embodiments, the amount of cellular material is no more than about 1 microgram. In still further or additional embodiments, the amount of cellular material is no more than about 1 gram.

In further or additional embodiments, the amount of cellular material is from about 1 picogram to about 1 gram. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms. In additional embodiments, the cellular material comprises an amount that is from about 50 picograms to about 1 micrograms, from about 100 picograms to about 500 picograms, from about 200 picograms to about 500 picograms, from about 500 picograms to about 1 nanograms, from about 500 picograms to about 500 nanograms, or from about 1 nanograms to about 500 nanograms.

In further or additional embodiments, the amount of cellular material, including nucleic acids such as RNA or DNA, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms. In some embodiments, the amount of cellular material, including nucleic acids such as RNA or DNA, comprises an amount that is from about 1 picograms to about 1 micrograms, from about 100 picograms to about 500 picograms, from about 200 picograms to about 500 picograms, from about 500 picograms to about 1 nanograms, from about 500 picograms to about 500 nanograms, or from about 1 nanograms to about 500 nanograms.

In further or additional embodiments, the amount of cellular material, including nucleic acids such as RNA or DNA, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In some embodiments, isolated RNA from a collected skin sample is reverse transcribed into cDNA, for example for amplification by PCR to enrich for target genes. The expression levels of these target genes are quantified by quantitative PCR in a gene expression test. In some instances, in combination with quantitative PCR, a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as skin cancer. In some embodiments, a software program or module scores a sample based on gene expression levels. In some embodiments, the sample score is compared with a reference sample score to determine if there is a statistical significance between the gene expression signature and a disease.

In some instances, the layers of skin include epidermis, dermis, or hypodermis. The outer layer of epidermis is the stratum corneum layer, followed by stratum lucidum, stratum granulosum, stratum spinosum, and stratum basale. In some instances, the skin sample is obtained from the epidermis layer. In some cases, the skin sample is obtained from the stratum corneum layer. In some instances, the skin sample is obtained from the dermis.

In some instances, cells from the stratum corneum layer are obtained, which comprises keratinocytes. In some cases, melanocytes are not obtained from the skin sample.

Following extraction of nucleic acids from a biological sample, the nucleic acids, in some instances, are further purified. In some instances, the nucleic acids are RNA. In some instances, the nucleic acids are DNA. In some instances, the RNA is human RNA. In some instances, the DNA is human DNA. In some instances, the RNA is microbial RNA. In some instances, the DNA is microbial DNA. In some instances, human nucleic acids and microbial nucleic acids are purified from the same biological sample. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, or more than 10 μm.

In some cases, a yield of the nucleic acids products obtained using methods described herein is about 500 picograms or higher, about 600 picograms or higher, about 1000 picograms or higher, about 2000 picograms or higher, about 3000 picograms or higher, about 4000 picograms or higher, about 5000 picograms or higher, about 6000 picograms or higher, about 7000 picograms or higher, about 8000 picograms or higher, about 9000 picograms or higher, about 10000 picograms or higher, about 20000 picograms or higher, about 30000 picograms or higher, about 40000 picograms or higher, about 50000 picograms or higher, about 60000 picograms or higher, about 70000 picograms or higher, about 80000 picograms or higher, about 90000 picograms or higher, or about 100000 picograms or higher.

In some cases, a yield of the nucleic acids products obtained using methods described herein is about 100 picograms, 500 picograms, 600 picograms, 700 picograms, 800 picograms, 900 picograms, 1 nanogram, 5 nanograms, 10 nanograms, 15 nanograms, 20 nanograms, 21 nanograms, 22 nanograms, 23 nanograms, 24 nanograms, 25 nanograms, 26 nanograms, 27 nanograms, 28 nanograms, 29 nanograms, 30 nanograms, 35 nanograms, 40 nanograms, 50 nanograms, 60 nanograms, 70 nanograms, 80 nanograms, 90 nanograms, 100 nanograms, 500 nanograms, or higher.

In some cases, methods described herein provide less than less than 10%, less than 8%, less than 5%, less than 2%, less than 1%, or less than 0.5% product yield variations between samples.

In some cases, methods described herein provide a substantially homogenous population of a nucleic acid product.

In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. In some instances, the nucleic acids to be amplified are RNA including, but not limited to, human RNA and human microbial RNA. In some instances, the nucleic acids to be amplified are DNA including, but not limited to, human DNA and human microbial DNA. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

Provided herein are methods for detecting an expression level of one or more genes of interest from nucleic acids isolated from a biological sample. In some instances, the expression level is detected following an amplification reaction. In some instances, the nucleic acids are RNA. In some instances, the RNA is human RNA. In some instances, the RNA is microbial RNA. In some instances, the nucleic acids are DNA. In some instances, the DNA is human DNA. In some instances, the DNA is microbial DNA. In some instances, the expression level is determined using PCR. In some instances, the expression level is determined using qPCR. In some instances, the expression level is determined using a microarray. In some instances, the expression level is determined by sequencing.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### Markers

Interleukin 17A (IL-17A), also known as IL-17, CTLA-8 or Cytotoxic T-Lymphocyte-Associated Protein 8, is a proinflammatory cytokine produced by activated T cells. This cytokine regulates the activities of NF-kappaB and mitogen-activated protein kinases and can stimulate the expression of IL6 and cyclooxygenase-2 (PTGS2/COX-2), as well as enhance the production of nitric oxide (NO). In some instances, IL-17A has Gene ID: 3605.

Interleukin 22 (IL-22), also known as Cytokine Zcyto18, IL-TIF, IL-D110, or TIFa, is a member of the IL10 family of cytokines that mediate cellular inflammatory responses. The encoded protein functions in antimicrobial defense at mucosal surfaces and in tissue repair. This protein also has pro-inflammatory properties and plays a role in in the pathogenesis of several intestinal diseases. In some instances, IL-22 has Gene ID: 50616.

Interleukin 23 Subunit Alpha (IL-23A), also known as IL-23, SGRF, or P19, encodes a subunit of the heterodimeric cytokine interleukin 23 (IL23). IL23 is composed of this protein and the p40 subunit of interleukin 12 (IL12B). The receptor of IL23 is formed by the beta 1 subunit of IL12 (IL12RB1) and an IL23 specific subunit, IL23R. Both IL23 and IL12 can activate the transcription activator STAT4, and stimulate the production of interferon-gamma.

S100 Calcium Binding Protein A7 (S100A7), also known as PSOR1 or Psoriasin, encodes a protein that is a member of the S100 family of proteins containing 2 EF-hand calcium-binding motifs. S100 proteins are localized in the cytoplasm and/or nucleus of a wide range of cells, and involved in the regulation of a number of cellular processes such as cell cycle progression and differentiation. The protein is overexpressed in hyperproliferative skin diseases, exhibits antimicrobial activities against bacteria and induces immunomodulatory activities. In some instances, S100A7 has Gene ID: 6278.

S100 Calcium Binding Protein A8 (S100A8), also known as Leukocyte L1 Complex Light Chain, Cystic Fibrosis Antigen, or Calgranulin A, encodes a protein that is a member of the S100 family of proteins containing 2 EF-hand calcium-binding motifs. This protein may function in the inhibition of casein kinase and as a cytokine. Altered expression of this protein is associated with the disease cystic fibrosis. In some instances, S100A8 has Gene ID: 6279.

S100 Calcium Binding Protein A9 (S100A9), also known as MRP-14, CAGB, or L1AG, encodes a protein that is a member of the S100 family of proteins containing 2 EF-hand calcium-binding motifs. S100 proteins are localized in the cytoplasm and/or nucleus of a wide range of cells, and involved in the regulation of a number of cellular processes such as cell cycle progression and differentiation. S100 genes include at least 13 members which are located as a cluster on chromosome 1q21. This antimicrobial protein exhibits antifungal and antibacterial activity. In some instances, S100A9 has Gene ID: 6280.

Thymic Stromal Lymphopoietin (TSLP) encodes a hemopoietic cytokine proposed to signal through a heterodimeric receptor complex composed of the thymic stromal lymphopoietin receptor and the IL-7R alpha chain. It mainly impacts myeloid cells and induces the release of T cell-attracting chemokines from monocytes and enhances the maturation of CD11c(+) dendritic cells. The protein promotes T helper type 2 (TH2) cell responses that are associated with immunity in various inflammatory diseases, including asthma, allergic inflammation and chronic obstructive pulmonary disease. In some instances, TSLP has Gene ID: 85480.

C-C Motif Chemokine Ligand 17 (CCL17), also known as Small-Inducible Cytokine A17, TARC, or ABCD-2, is one of several Cys-Cys (CC) cytokine genes clustered on the q arm of chromosome 16. The CC cytokines are proteins characterized by two adjacent cysteines. The cytokine encoded by this gene displays chemotactic activity for T lymphocytes, but not monocytes or granulocytes. The product of this gene binds to chemokine receptors CCR4 and CCR8. This chemokine plays important roles in T cell development in thymus as well as in trafficking and activation of mature T cells. In some instances, TSLP has Gene ID: 85480.

C-C Motif Chemokine Ligand 18 (CCL18), also known as SCYA18, CD-CK1, or AMAC1, is one of several Cys-Cys (CC) cytokine genes clustered on the q arm of chromosome 17. The CC cytokines are proteins characterized by two adjacent cysteines. The cytokine encoded by this gene displays chemotactic activity for naive T cells, CD4+ and CD8+ T cells and nonactivated lymphocytes, but not for monocytes or granulocytes. This chemokine attracts naive T lymphocytes toward dendritic cells and activated macrophages in lymph nodes. In some instances, CCL18 has Gene ID: 6362.

C-C Motif Chemokine Ligand 19 (CCL19), also known as CK Beta-11, Exodus-3, or MIP3B, is one of several CC cytokine genes clustered on the p-arm of chromosome 9. The CC cytokines are proteins characterized by two adjacent cysteines. The cytokine encoded by this gene may play a role in normal lymphocyte recirculation and homing. It also plays an important role in trafficking of T cells in thymus, and in T cell and B cell migration to secondary lymphoid organs. It specifically binds to chemokine receptor CCR7. In some instances, CCL19 has Gene ID: 6363.

C-C Motif Chemokine Ligand (CCL26), also known as Macrophage Inflammatory Protein 4-Alpha, Eotaxin-3, or SCYA26, is one of two Cys-Cys (CC) cytokine genes clustered on the q arm of chromosome 7. The CC cytokines are proteins characterized by two adjacent cysteines. The cytokine encoded by this gene displays chemotactic activity for normal peripheral blood eosinophils and basophils. The product of this gene is one of three related chemokines that specifically activate chemokine receptor CCR3. This chemokine may contribute to the eosinophil accumulation in atopic diseases. In some instances, CCL26 has Gene ID: 10344.

C-C Motif Chemokine Ligand (CCL27), also known as Skinkine, IL-11, Ralpha-Locus Chemokine, or CTACK, is one of several CC cytokine genes clustered on the p-arm of chromosome 9. The CC cytokines are proteins characterized by two adjacent cysteines. The protein encoded by this gene is chemotactic for skin-associated memory T lymphocytes. This cytokine may also play a role in mediating homing of lymphocytes to cutaneous sites. It specifically binds to chemokine receptor 10 (CCR10). Studies of a similar murine protein indicate that these protein-receptor interactions have a pivotal role in T cell-mediated skin inflammation. In some instances, CCL27 has Gene ID: 10850.

C-X-C Motif Chemokine Ligand 9 (CXCL9), also known as Small-Inducible Cytokine B9, SCYB9, Crg-10, or HuMIG, encodes a protein thought to be involved in T cell trafficking. The encoded protein binds to C-X-C motif chemokine 3 and is a chemoattractant for lymphocytes but not for neutrophils. In some instances, CXCL9 has Gene ID: 4283.

C-X-C Motif Chemokine Ligand 10 (CXCL10), also known as Gamma IP10, SCYB10, or Crg-2, encodes a chemokine of the CXC subfamily and ligand for the receptor CXCR3. Binding of this protein to CXCR3 results in pleiotropic effects, including stimulation of monocytes, natural killer and T-cell migration, and modulation of adhesion molecule expression. In some instances, CXCL10 has Gene ID: 3627.

C-X-C Motif Chemokine Ligand 11 (CXCL11), also known as Beta-R1, SCYB11, or ITAC, is a CXC member of the chemokine superfamily. Its encoded protein induces a chemotactic response in activated T-cells and is the dominant ligand for CXC receptor-3. IFN-gamma is a potent inducer of transcription of this gene. In some instances, CXCL11 has Gene ID: 6373.

Interleukin 13 (IL-13) encodes an immunoregulatory cytokine produced primarily by activated Th2 cells. This cytokine is involved in several stages of B-cell maturation and differentiation. It up-regulates CD23 and MHC class II expression, and promotes IgE isotype switching of B cells. This cytokine down-regulates macrophage activity, thereby inhibits the production of pro-inflammatory cytokines and chemokines. This cytokine is found to be critical to the pathogenesis of allergen-induced asthma but operates through mechanisms independent of IgE and eosinophils. This gene, IL3, IL5, IL4, and CSF2 form a cytokine gene cluster on chromosome 5q, with this gene particularly close to IL4. In some instances, IL-13 has Gene ID: 3596.

Interleukin 13 Receptor (IL-13R) is a type I cytokine receptor, binding Interleukin-13. It consists of two subunits, encoded by IL13RA1 and IL4R, respectively. These two genes encode the proteins IL-13Rα1 and IL-4Rα. These form a dimer with IL-13 binding to the IL-13Rα1 chain and IL-4Rα stabilises this interaction.

Interleukin 31 (IL-31), also known as IL-31, which is made principally by activated Th2-type T cells, interacts with a heterodimeric receptor consisting of IL31RA (MIM 609510) and OSMR (MIM 601743) that is constitutively expressed on epithelial cells and keratinocytes. IL31 may be involved in the promotion of allergic skin disorders and in regulating other allergic diseases, such as asthma. In some instances, IL-31 has Gene ID: 386653.

Interleukin 4 Receptor (IL4R), mediates enhanced glucose and glutamine metabolism in breast cancer cells. In some instance, IL4R has Gene ID: 3566.

Nitric Oxide Synthetase 2 (NOS2), also known as Inducible NOS2 or Hepatocyte NOS, encodes a nitric oxide synthase which is expressed in liver and is inducible by a combination of lipopolysaccharide and certain cytokines. In some instances, NOS2 has Gene ID: 4843.

### Example Normalized Expression Values and Ratios

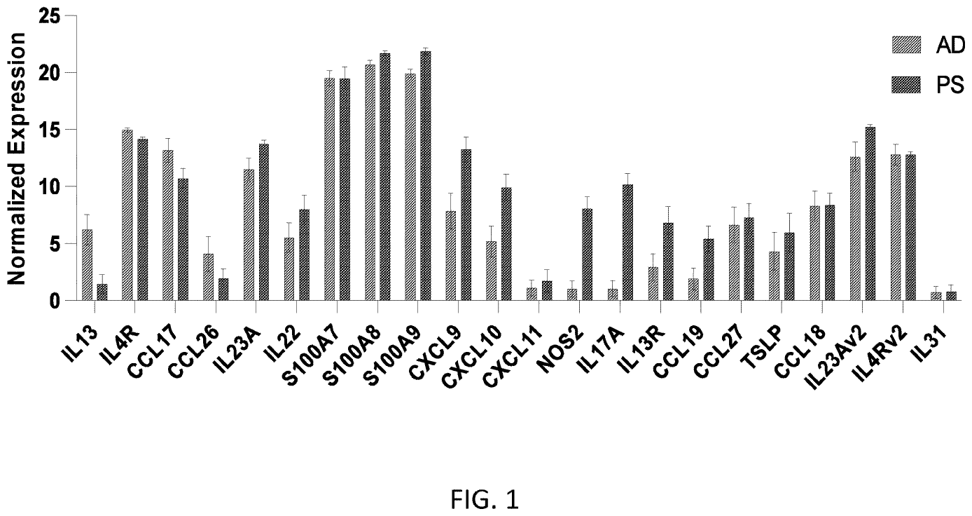

FIG. 1 depicts normalized expression levels for a plurality of genes. In this depicted example, the plurality of genes includes IL13, IL4R, CCL17, CCL26, IL23A, IL22, S100A7, S100A8, S100A9, CXCL9, CXCL10, CXCL 11, NOS2, IL17A, IL13R, CCL19, CCL27, TSLP, CCL18, IL23A, IL4R, and IL31. In some embodiments, the expression values of the plurality of genes are normalized with respect to a housekeeping gene, such as Actin Beta (ACTB).

The normalized expression values depicted are calculated based on determined gene expression levels from samples of patient afflicted with atopic dermatitis and/or psoriasis. In some embodiments, the gene expression levels are based on delta CT values for each gene. For each gene shown, two expression levels, each with a confidence value, are depicted. The left expression level for each gene depicts the expression level found in patients with atopic dermatitis, while the right expression level for each gene depicts the expression level found in patients with psoriasis. Accordingly, when a sample is taken from a patient according to the methods described above, if the sample is determined to have a normalized expression level of a certain gene (e.g., IL13) within the confidence intervals of the left expression level associated with the certain gene, the normalized expression level indicates that the subject has atopic dermatitis. Conversely when a sample is taken from a patient according to the methods described above, if the sample is determined to have a normalized expression level of a certain gene (e.g., IL17A) within the confidence intervals of the right expression level associated with the certain gene, the normalized expression level indicates that the subject has psoriasis.

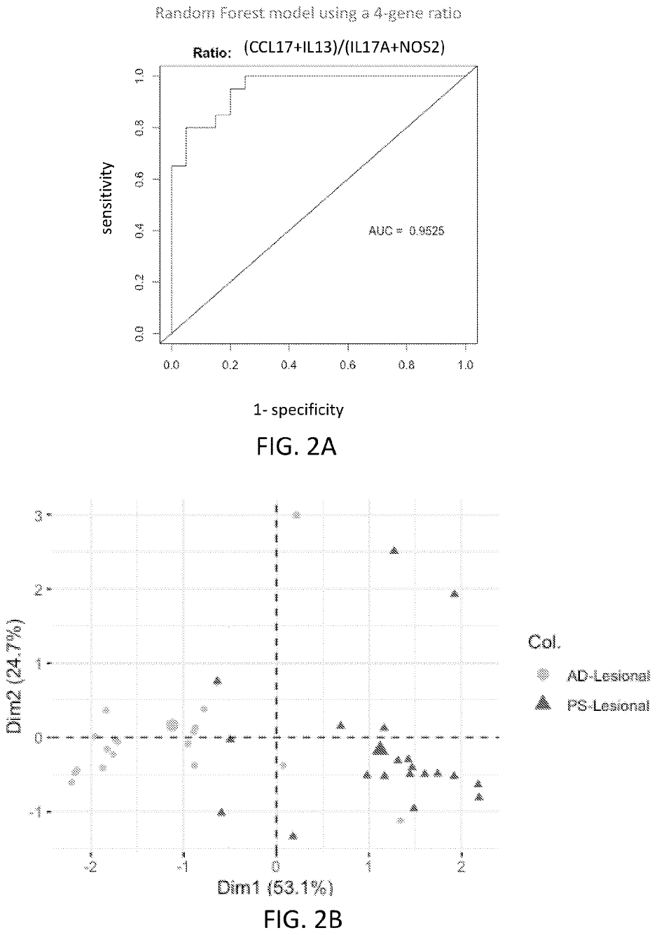

Thus, by receiving a sample and determining the normalized gene expression levels, each gene expression level may be classified as indicating that the patient has, is likely to have, is unlikely to have, or does not have atopic dermatitis or psoriasis based on where the normalized gene expression level falls with regard to the confidence intervals (as described further with respect to FIGS. 2-7).

FIG. 2A depicts an AUC (“area under curve”) as an accuracy measure for an example ratio (also referred to as an “indicator value”) calculated using machine learning model based on a plurality of gene expression levels determined based on samples received from subjects with atopic dermatitis or psoriasis. As described herein, a “ratio” is a value determined based on a comparison of one or more expression levels for one or more genes. A ratio may be calculated upon receiving one or more expression levels for one or more genes from a subject. As further described herein, a ratio may be compared to an indicator value, wherein the indicator value is additionally calculated based on the one or more expression levels for the one or more genes from a plurality of subject. For example, based on the one or more expression levels for a plurality of subjects, an indicator value may be determined to be 1.0. One or more expression levels may then be received for a subject, and a ratio may be determined for that subject. The ratio then be compared to the indicator value to determine if the subject has one or more conditions associated with the ratio and the indicator value. Additionally, the ratio and diagnosis of the one or more conditions may be used to adjust the indicator value. Accordingly, in some embodiments, the indicator value may be a value determined based on one or more ratios.

In this depicted example, the indicator value was calculated based on normalized expression levels of IL17A, NOS2, CCL17, and IL13 in sample from twenty patients who had atopic dermatitis and twenty patients who had psoriasis. In some embodiments, the machine learning model is a regression model. In some embodiments, the regression model is a logistics regression model. In some embodiments, the machine learning model may be a random forest model. In this depicted example, the “Area Under Curve”, which estimates the accuracy of the ratio in differentiating atopic dermatitis from psoriasis is 0.9525. Thus, if a sample is received from a subject, and the sum of the normalized expression values of CCL17 and IL13 divided by the sum of the normalized expression values of IL17A and NOS2 (e.g., the ratio determined using IL17A, NOS2, CCL17, and IL13) from the sample is above the defined indicator value (e.g., 1.0), the subject more likely has atopic dermatitis than psoriasis, and if the sum of the normalized expression values of CCL17 and IL13 divided by the sum of the normalized expression values of IL17A and NOS2 (e.g., the ratio determined using IL17A, NOS2, CCL17, and IL13) from the sample is below the indicator value, the subject is more likely to have psoriasis than atopic dermatitis. The accuracy of the prediction using the ratio affects the AUC, which can be used to adjust the parameters of the machine learning model in determining how to weight one or more genes or what the ratio used should be. For example, the normalized expression level for certain genes (e.g., CCL17) may be multiplied by a weight coefficient when determining the ratio or an indicator value.

FIG. 2B depicts separation of disease classes using the IL17A, NOS2, CCL17, and IL13 genes grouped by principal competent analysis.

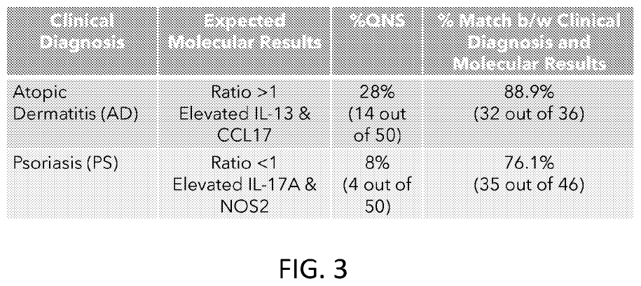

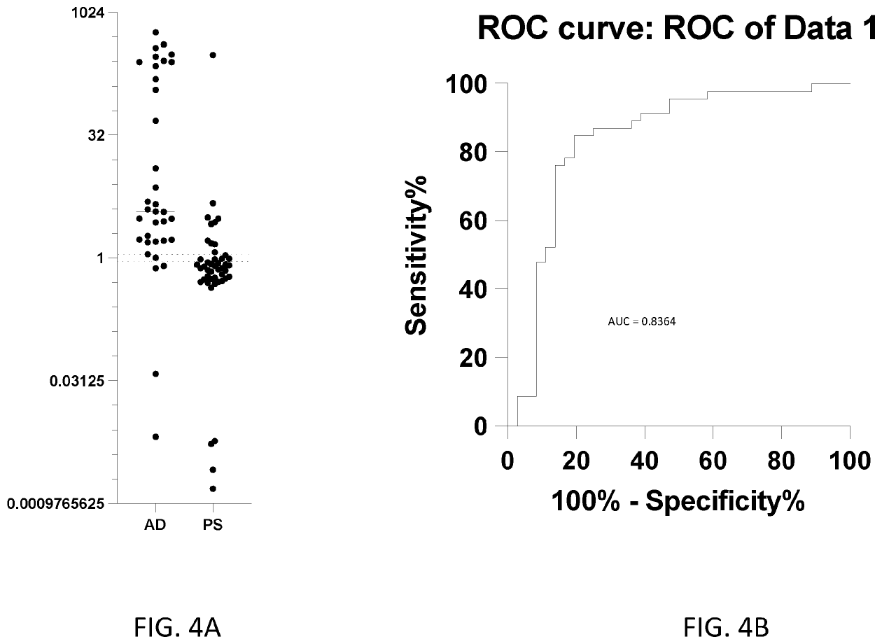

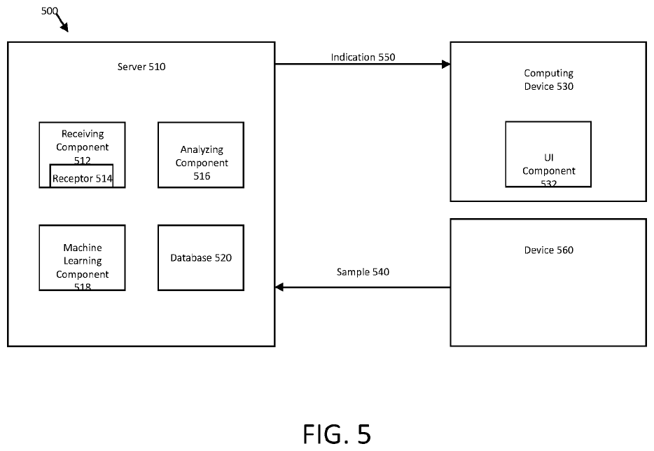

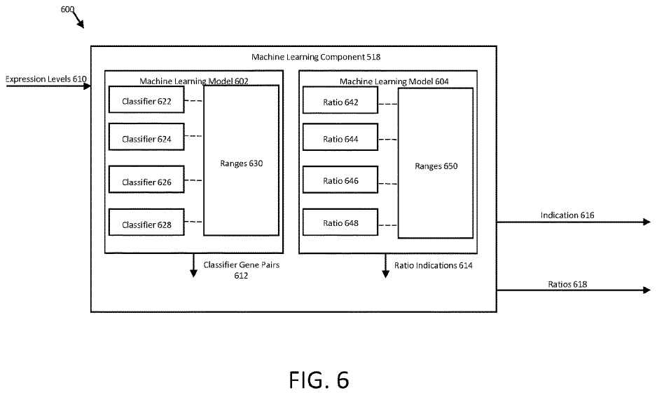

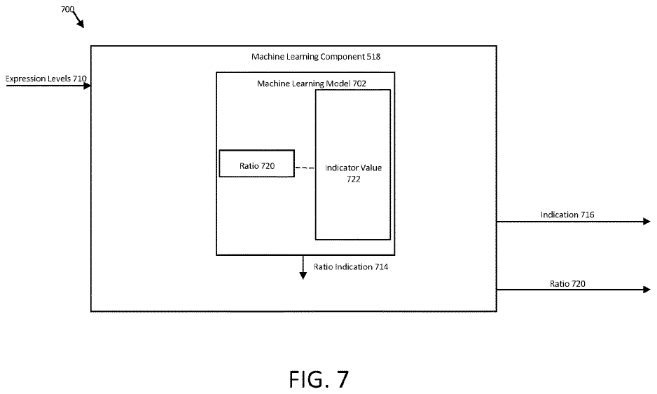

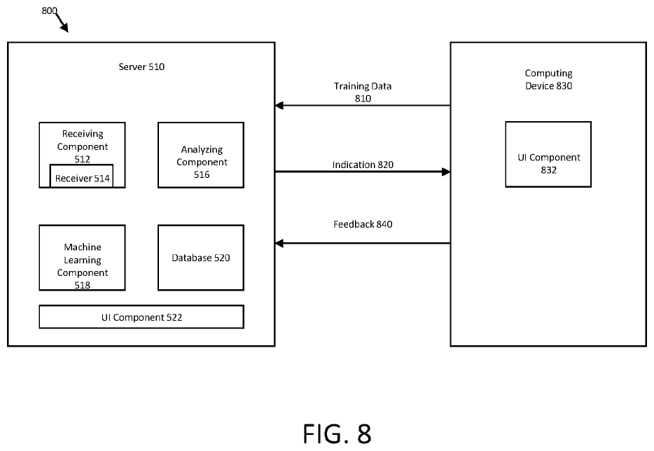

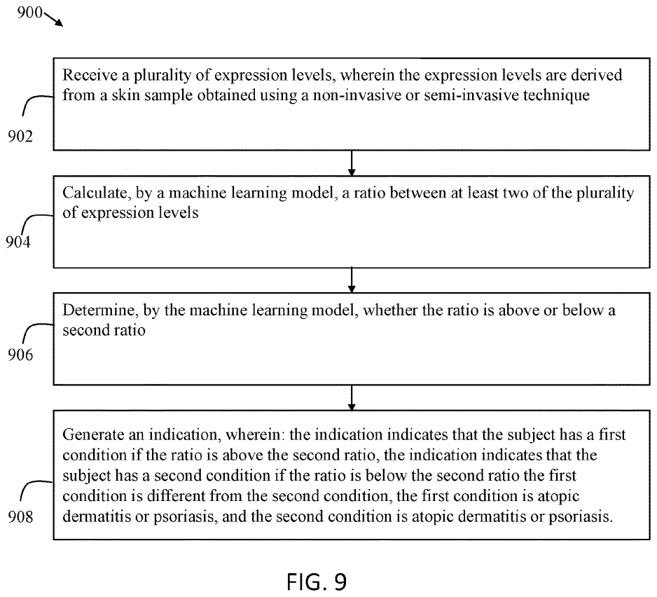

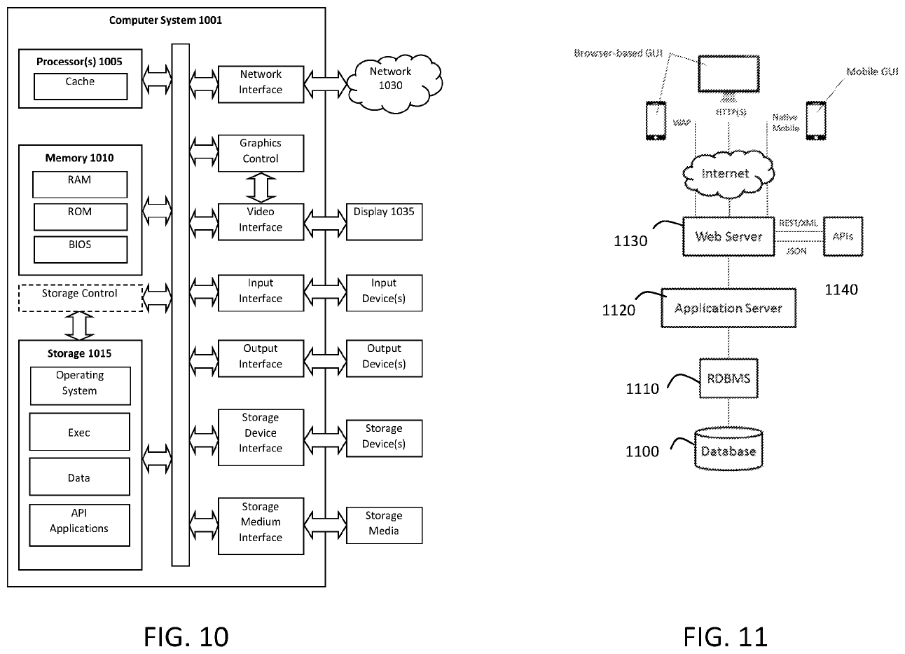

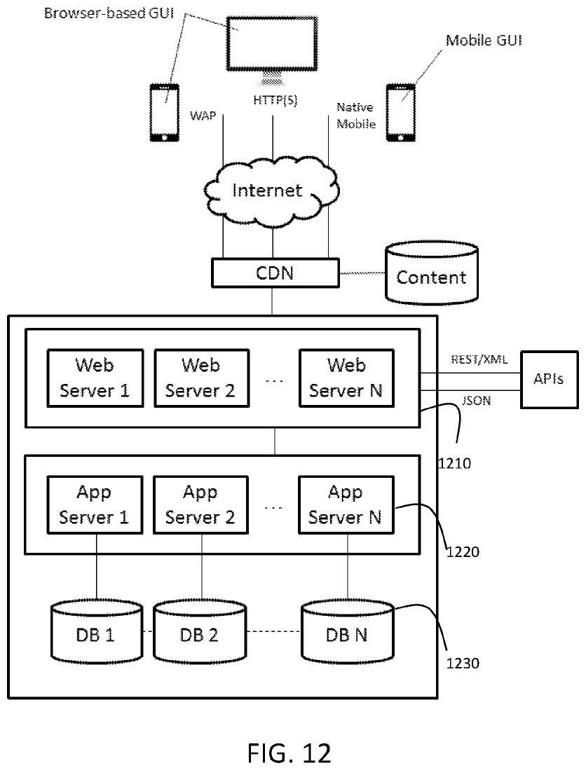

FIG. 3 depicts example data using a ratio calculated by the machine learning model of FIG. 2. In this depicted example, an indicator value of 1.0 is used (e.g., the indicator value being calculated based on dividing the sum of the normalized expression values of CCL17 and IL13 by the sum of the normalized expression values of IL17A and NOS2 for a plurality of subjects and weighting the data to determine the indicator value of 1.0). In this depicted embodiment, ratios were calculated for each subject in order to determine if the subject had atopic dermatitis or psoriasis by comparing each ratio to the indicator value of 1.0. In some embodiments, the ratios may be provided to the machine learning model as feedback, and the machine learning model may have one or more parameters adjusted as a result. In some embodiments, the machine learning model may adjust the indicator value based on the received ratios.

In this depicted example, samples were received from 36 patients that were diagnosed with atopic dermatitis. The gene expression levels were determined for each sample of the 36 patients, and the ratio for each patient was calculated. The ratio for 32 of the 36 patients diagnosed with atopic dermatitis was above 1.0 (e.g., the indicator value in this depicted example), and the ratio for the remaining 4 of the 36 patients diagnosed with atopic dermatitis was below 1.0, showing a 88.9% success rate in predicting atopic dermatitis using the ratio.

Similarly, in this depicted example, samples were received from 46 patients that were diagnosed with psoriasis. The gene expression levels were determined for each sample of the 36 patients, and the ratio for each patient was calculated. The ratio for 35 of the 46 patients diagnosed with psoriasis was below 1.0, and the ratio for the remaining 11 of the 46 patients diagnosed with psoriasis was below 1.0, showing a 76.1% success rate in predicting atopic dermatitis using the ratio.

FIG. 4A depicts example data for the 36 subjects diagnosed with atopic dermatitis and the 46 subjects diagnosed with psoriasis as described with respect to FIG. 3. The left portion of data indicates the value of each ratio determined for the 36 subjects diagnosed with atopic dermatitis, while the right portion of data indicates the value of each ratio determined for the 46 subjects diagnosed with psoriasis.

FIG. 4B depicts a revised AUC as a measure of accuracy of diagnosis based on the indicator value based on new ratio data. The ratios as determined as described with respect to FIG. 4A were provided to the machine learning model as feedback, in order to allow the machine learning model to provide an indicator value based on a more complete data set. Additionally, after receiving the data, the a new AUC of 0.8364 for using an indicator value of 1.0 calculated by using the same genes (e.g., IL17A, NOS2, CCL17, and IL13).

Thus, as described above, the machine learning model as described with respect to FIGS. 2-4 can be used to indicate whether a subject more likely has atopic dermatitis or psoriasis based on a ratio between normalized gene expression levels determined from a sample of the patient. While IL17A, NOS2, CCL17, and IL13 are used to determine the ratios for each subject, and the indicator value that the ratios are compared to, as described above, these genes are exemplary, and other genes may be used in determining ratios for differentiating atopic dermatitis from psoriasis, or other diseases from one another.

Accordingly, by knowing the normalized expression values after receiving a sample from a patient, a patient can be accurately diagnosed as having atopic dermatitis instead of psoriasis, or psoriasis instead of atopic dermatitis. Importantly, determining a ratio to indicate whether the patient has atopic dermatitis or psoriasis does not require a control sample (e.g., from non-lesional skin) because the ratio can be determined from the expression levels of just a lesional sample. Even further, by utilizing a machine learning model, the process of determining the ratio has an increased efficiency and decreased time requirements (e.g., taking minutes or seconds) compared to determining the ratio manually, which could manually require additionally resources and could take months or days.

Additionally, in some embodiments, a range of ratios (also referred to as a range of “indicator values” with respect to FIGS. 5-6 and 8) may be used ratio than a single value for the ratio.

While the indicator value of 1.0 is described above for the genes described above, one of skill in the art would understand that the different genes may be used, and consequently, the indicator value may be a different value that 1.0.

### Example Systems for Diagnosing Atopic Dermatitis and/or Psoriasis

FIG. 5 depicts an example computer system 500 for diagnosing atopic dermatitis, psoriasis, or both, in a human subject. In this depicted example, system 500 includes server 510, computing device 530, and device 560.

In this depicted example, server 510 includes receiving component 512, analyzing component 516, machine learning component 518, database 520, and user interface (UI) component 522.

In this depicted example, receiving component 512 further includes receptor 514. In some embodiments, server 510 may be configured to receive one or more samples (e.g., skin samples from the human subject) at receptor 514. Receptor 514 may be one or more physical devices as described above, or any combination thereof. In some embodiments, server 510 may not include receptor 514 and may instead receive data associated with the one or more samples to be analyzed by analyzing component 516.

In this depicted example, analyzing component 516 may analyze the received one or more samples. For example, analyzing component may determine expression levels of one or more genes in the one or more samples. In some embodiments, the one or more genes may include IL13, IL4R, CCL17, CCL26, IL23A, IL22, S100A7, S100A8, S100A9, CXCL9, CXCL10, CXCL11, NOS2, IL17A, IL13R, CCL19, CCL27, TSLP, CCL18, IL23A, IL4R, and/or IL31 genes or combinations thereof. In some embodiments, the analyzing component 516 may receive the expression levels of the one or more genes. In some embodiments, the analyzing component 516 normalizes the expression levels of the one or more genes with respect to ACTB.

In this depicted example, the machine learning component 518 may determine one or more classifiers and/or ratios based on the expression levels of the one or more genes (as described further with respect to FIG. 6). In some embodiments, the machine learning component 518 may further determine indicator values based on received ratios as training data (as further described with respect to FIG. 7). In some embodiments, machine learning component 518 may further adjust indicator values based on newly calculated ratios. In some embodiments, the machine learning component 518 may include one or more machine learning models.

In some embodiments, machine learning component 518 may include a first machine learning model. In some embodiments, the first machine learning model may receive the expression levels of the one or more genes as input, and may determine a classifier for each of the expression levels indicating whether the subject has, is likely to have, is unlikely to have, or does not have a condition (e.g., atopic dermatitis or psoriasis). Additionally, in some embodiments, each classifier may be associated with one of the one or more genes. For example, the first machine learning model may receive expression levels for each of the IL13, IL4R, CCL17, CCL26, IL23A, IL22, S100A7, S100A8, S100A9, CXCL9, CXCL10, CXCL11, NOS2, IL17A, IL13R, CCL19, CCL27, TSLP, CCL18, IL23A, IL4R, and/or IL31 genes, and may determine a classifier to each of the genes. While certain genes are described above, these genes are exemplary, and other genes not listed may be used instead or in conjunction with the genes listed above. In some embodiments, the machine learning component 518 may normalize the one or more expression levels. In some embodiments, the analyzing component 516 may normalize the one or more expression levels before the machine learning component receives the normalized one or more expression levels. In some embodiments, analyzing component 516 may receive expression values that have already been normalized.

The classifiers may be determined based on whether the expression level of the gene indicates that the subject has one of more conditions. In some embodiments, if the expression level for a particular gene falls within a first range, the expression level may indicate that the subject has a first condition (e.g., atopic dermatitis), and if the expression level for the particular gene falls within a second range, the expression level may indicate that the subject has a second condition (e.g., psoriasis). In some embodiments, the first range and the second range may overlap. In some embodiments, the first range and the second range may not overlap. Thus, in some embodiments, if the machine learning model determines that (1) the expression level falls in the first range, it may assign a first classifier to the associated gene indicating that the subject likely has atopic dermatitis, (2) the expression level falls out of the first range, it may assign a second classifier to the associated gene indicating that the subject likely does not have atopic dermatitis, (3) the expression level falls in the second range, it may assign a third classifier to the gene indicating that the subject likely has psoriasis, and (4) the expression level falls out of the second range, it may assign a fourth classifier to the gene indicating that the subject likely does not have psoriasis. The machine learning model may assign a classifier to one or more genes based on the sample. While certain classifiers are described above (e.g., indicating the subject likely has atopic dermatitis, indicating that the subject does not likely have atopic dermatitis, indicating that the subject likely has psoriasis, and indicating that the subject does not likely have psoriasis), these classifiers are exemplary, and other classifiers may be used. For example, other classifiers associated with other conditions may be used. As another example, other classifiers associated with different degrees (e.g., very likely, not very likely, extremely likely, or extremely unlikely) may be used. In some embodiments, the first machine learning model may receive feedback regarding the classifiers, and may adjust one or more parameters of the first machine learning model in response to the feedback. In some embodiments, the feedback may include one or more corrected classifiers.

The machine learning component 518 may further determine one or more ratios between gene expression levels and compare the one or more ratios to one or more indicator values in order to determine if the patient has one or more of the conditions described above. In some embodiments, the one or more indicator values may be received by machine learning component 518. In some embodiments, the one or more indicator values are determined by the machine learning component 518. In some embodiments, the machine learning component 518 may determine the one or more ratios to determine if the patient has the one or more conditions using a second machine learning model. In some embodiments, the second machine learning model may receive the expression levels as described above as input, may determine the one or more ratios for the subject's sample based on the expression levels, and may provide an indication of whether the patient has, is likely to have, is unlikely to have, or does not have the one or more conditions (as described further with respect to FIG. 6) based on comparing the one or more ratios to one or more indicator values. In some embodiments, a ratio may be determined by comparing the expression level of a first gene to the expression level of a second gene from the sample. In some embodiments, a ratio may be determined by comparing the expression level of a first gene and a second gene to the expression level of a third gene and a fourth gene (as described with respect to FIG. 2). In some embodiments, a ratio may be determined by comparing the expression level of a first gene to the expression level of a second gene and a third gene. In some embodiments, a ratio may be determined by comparing multiple expression levels associated with a first plurality of genes to multiple expression levels associated with a second plurality of genes. In some embodiments, a ratio may indicate that the subject has, is likely to have, is unlikely to have, or does not have atopic dermatitis. In some embodiments, a ratio may indicate that the subject has, is likely to have, is unlikely to have, or does not have psoriasis. In some embodiments, the ratio may indicate that the subject has, is likely to have, is unlikely to have, or does not have a condition by comparing the ratio determined by the machine learning model to one or more indicator values, where the one or more indicator values was determined using one or more separate sets of expression levels associated with the same genes of the ratio (e.g., by using previously determined ratio data to determine the one or more indicator values).

For example, a ratio determined by using the expression levels of IL13 and CXCL9 to the expression levels of NOS2 and IL17A from the sample may be compared to an indicator value or a set of indicator values determined by comparing expression levels of IL13 and CXCL9 to NOS2 and IL17A from previous samples. For example, if the normalized expression levels for IL13, CXCL9, NOS2, and IL17A are 5.4, 7.2, 0.5, and 0.3, the ratio may be determined as 15.75 (e.g., by dividing the sum of the expression levels of IL13 and CXCL9 by the sum of expression levels for NOS2 and IL17A). The ratio may then be compared to the set of indicator values. In this particular embodiment, the set of indicator values may include values between 5.5 and 82.5 as well as values between 0.454 and 0.780. In this particular embodiment, the values between 5.5 and 82.5 indicate that a subject has or is likely to have atopic dermatitis, while the values between 0.454 and 0.780 indicate that subject has or is likely to have psoriasis. In this particular example, the ratio determined by the second machine learning model is 15.75, and thus falls in the set of indicator values between 5.5 and 82.5, indicating the subject has or is likely to have atopic dermatitis rather than psoriasis. Alternatively, in some embodiments, the ratio may be compared to a single indicator value (e.g., 12.0), and the subject may be indicated as having atopic dermatitis or psoriasis if the ratio is above or below the single indicator value. While certain values in the set of indicator values are described above, these values are exemplary and other values may be used. In some embodiments, the ranges of indicator values that indicate that a subject may have atopic dermatitis or psoriasis may overlap for specific ratios. Additionally, while the genes and ratios described above include expression values for IL13, CXCL9, NOS2, and IL17A, different genes and ratios can be used. For example, other genes that may be used in determine ratios may be only IL13 and IL17A, CXCL9 and IL17A, CLCL10 and CXCL9, or IL13R and CCL26.

In some embodiments, a ratio may not fall within a set of indicator values. In those embodiments, the ratio may not indicate that the subject has or is likely to have atopic dermatitis or psoriasis. In some embodiments, a plurality of ratios may be determined. In some embodiments, one or more of the plurality of ratios may indicate that the subject has or is likely to have atopic dermatitis. In some embodiments, one or more of the plurality of ratios is likely to have psoriasis. In some embodiments, one or more ratios may indicate that the subject has or is likely to have atopic dermatitis and one or more ratios may indicate that the subject has or is likely to have psoriasis. In some embodiments, none of the ratios may indicate that the subject has or is likely to have atopic dermatitis and one or more ratios may indicate that the subject has or is likely to have psoriasis.

Alternatively, in embodiments, where the ratio may be compared to a single indicator value, the ratio may be determined as either above or below the single indicator value. In some embodiments, if the ratio is determined to be above the single indicator value, the subject may be determined to have atopic dermatitis instead of psoriasis. In some embodiments, if the ratio is determined to be above the single indicator value, the subject may be determined to have psoriasis instead of atopic dermatitis. In some embodiments, if the ratio is determined to be below the single indicator value, the subject may be determined to have atopic dermatitis instead of psoriasis. In some embodiments, if the ratio is determined to be below the single indicator value, the subject may be determined to have psoriasis instead of atopic dermatitis.

In some embodiments, sets of indicator values or single indicator value may be stored in database 520. In some embodiments, machine learning component 518 may retrieve the indicator values from database 520. In some embodiments, the one or more classifiers may be stored in database 520. In some embodiments, machine learning component 518 may retrieve the indicator values from database 520.

Thus, the machine learning model 518 may receive the expression levels, and may provide them as input to the first machine learning model and the second machine learning model. The first machine learning model may receive the expression levels for each of the genes, and assign a classifier to each expression level, indicating whether each gene suggests that the subject has, is likely to have, is unlikely to have, or does not have at least one condition of the one or more conditions. The second machine learning model may receive the expression levels for each of the genes, and may calculate one or more ratios based on the expression levels in order to determine if the ratios fall within ranges in sets of indicator values indicating that the subject may have one or more conditions. Based on the one or more classifiers and the one or more ratios, the machine learning component 518 may determine whether the subject has the one or more conditions, and may generate an indicator 550 indicating whether the subject has the one or more conditions.

The server 510 may further be configured to provide the indication 550 to computing device 530. In this depicted embodiment, computing device 530 may further include UI component 532, which may display the indication 550, thereby allowing a user of computing device 530 to view the indication.

In some embodiments, the machine learning component 518 includes the first machine learning model and does not include the second machine learning model. In those embodiments, the indication 550 is generated based on the one or more classifiers for the one or more genes. In some embodiments, the machine learning component 518 includes the second machine learning model and does not include the first machine learning model. In those embodiments, the indication 550 is generated based on the one or more ratios.

In some embodiments, as described above with respect to FIGS. 2-4, the second machine learning model may use a single indicator value instead of a range of indicator values (e.g., the ratio of 0.9525 of FIG. 2 of the ratio of 0.8364 as described with respect to FIG. 4B). In those embodiments, the ratio determined based on the sample may be compared to the single indicator value. In some embodiments, if the ratio is higher than the single indicator value, the second machine learning model may provide an indication that the subject has a first condition (e.g., atopic dermatitis) instead of a second condition (e.g., psoriasis). In some embodiments, if the ratio is higher than the single indicator value, the second machine learning model may provide an indication that the subject has a second condition (e.g., psoriasis) instead of a first condition (e.g., atopic dermatitis).

In some embodiments, server 510 may not include UI component 522. In some embodiments, server 510 includes UI component 522. UI component 522 may be configured to display indication 550.

In some embodiments, the single indicator value or set of indicator values are determined by another machine learning model. In some embodiments, the another machine learning model is a regression model. In some embodiments, the another machine learning model may receive one or more ratios and one or more diagnoses (e.g., of whether a subject has atopic dermatitis or psoriasis based on comparing the one or more ratios to one or more indicator values) as feedback, and may adjust the single indicator value or the set of indicator values based on that feedback.

FIG. 6 shows an example process by machine learning component 518. In some embodiments, machine learning component 518 may include one or more machine learning models, as described with respect to FIG. 5. In this depicted embodiment, machine learning component 518 has a machine learning model 602 (e.g., the first machine learning model as described with respect to FIG. 5) and a machine learning model 604 (e.g., a second machine learning model as described with respect to FIG. 5).

In this depicted embodiment, machine learning component receives expression levels 610. Expression levels 610 includes a plurality of expression levels of genes, where the expression levels of genes are determined based on a sample taken from a subject. The genes may include one or more of the IL13, IL4R, CCL17, CCL26, IL23A, IL22, S100A7, S100A8, S100A9, CXCL9, CXCL10, CXCL11, NOS2, IL17A, IL13R, CCL19, CCL27, TSLP, CCL18, IL23A, IL4R, and/or IL31 genes.

In this depicted embodiment, machine learning model 602 receives expression levels 610 and determines classifier 622, classifier 624, classifier 626, classifier 628 based on the expression levels 610 and one or more ranges of ranges 630. The machine learning model 602 may determine each of the classifiers by comparing the expression level of a gene to one or more ranges of expression levels in ranges 630 associated with the gene, where each range of ranges 630 includes a range of expression levels for the gene indicating that a subject has or is likely to have a condition.

For example, a first expression level of expression levels 610 associated with a first gene may be compared to two ranges of expression levels of ranges 630 in order to determine if the first expression level falls within either of the two ranges of expression levels for the first gene. In some embodiments, the two ranges of the expression levels may overlap. Each of the two ranges may be associated with a condition, e.g., a first range of the two ranges may be associated with atopic dermatitis and a second range of the two ranges may be psoriasis. Based on if the expression level falls within each of the two ranges, the machine learning model 602 assigns a classifier indicating if the expression level of the first gene indicates that the subject has, is likely to have, is unlikely to have, or does not have the conditions associated with the one or more ranges. In this depicted example, classifier 622 indicates that the subject has or is likely to have atopic dermatitis and does not have or is not likely to have psoriasis, classifier 624 indicates that the subject has or is likely to have psoriasis and does not have or is not likely to have atopic dermatitis, classifier 626 indicates that the subject has or is likely to have both atopic dermatitis and psoriasis, and classifier 628 indicates that the subject does not have or is not likely to have either atopic dermatitis or psoriasis. While certain conditions and classifiers are listed above, these conditions and classifiers are exemplary, and other conditions and classifiers may be used.

Thus, in this depicted example, if the first expression level falls within the first range of the two ranges but not the second range of the two ranges, the machine learning model 602 may assign classifier 622 to indicate that the subject has or likely has atopic dermatitis but does not have or is not likely to have psoriasis because the expression level of the first gene falls in the first range but not in the second range. If the first expression level falls out of the first range but in the second range, the machine learning model 602 may assign classifier 624 to indicate that the subject does not have or does not likely have atopic dermatitis but does have or is likely to have psoriasis because the expression level of the first gene falls out the first range but does fall in the second range. If the first expression level falls within both the first range and the second range, the machine learning model 602 may assign classifier 626 to indicate that the subject has or likely has both atopic dermatitis and psoriasis because the expression level of the first gene falls in the first range and the second range, and if the first expression level falls out of both the first range and the second range, the machine learning model 602 may assign classifier 628 to indicate that the subject does not have or does not likely have either atopic dermatitis or psoriasis because the expression level of the first gene falls out the first range and the second range.

After assigning classifiers to the expression levels, the machine learning model 602 may pair a gene and the classifier associated with the gene to form a classifier gene pair of classifier gene pairs 612. Thus, for each expression level of expression levels 610, machine learning model 602 may assign a classifier indicating whether the patient has or is likely to have atopic dermatitis, psoriasis, both, or neither, and then may generate a classifier-gene pair to show what conditions the subject may have based on the expression level of the gene.

In some embodiments, the machine learning model 602 does not generate a classifier gene pair, and instead only provides the classifier for each gene.

In this depicted embodiment, machine learning model 604 receives expression levels 610 and determines ratio 642, ratio 644, ratio 646, and ratio 648 based on the received expression levels. The ratios 642, 644, 646, and 648 may be determined by dividing the normalized expression level of a first gene by the normalized expression level of second gene, by dividing the normalized expression level of a first gene by the sum of normalized expression levels of a second gene and a third gene, by dividing the sum of normalized expression levels of a first gene and a second gene by the sum of normalized expression levels of a third gene and a fourth gene, or by dividing the sum of normalized expression levels of a first gene and a second gene by a normalized expression ratio of a third gene. In some embodiments, a ratio may be determined by dividing the sum of normalized expression levels of four different genes by the sum of normalized expression ratios of four other genes, where the four other genes are different from the four different genes. In some embodiments, a ratio may be calculated by dividing a normalized expression level of one gene or a sum of normalized expression levels for any number of different genes by a normalized expression level of another gene or a sum of normalized expression levels for any number of other genes, wherein the other genes are not included in the different genes.

After determining ratios 642, 644, 646, and 648, machine learning model 604 may compare ratios 642, 644, 646, and 648 to one or more ranges of indicator values in ranges 650 (as described above with respect to FIG. 5). Each range of indicator values in ranges 650 indicates, when a ratio falls in the range of indicator values, whether a subject has a first condition (e.g., atopic dermatitis) instead of a second condition (e.g., psoriasis) or the second condition instead of the first condition. Thus, upon generating ratios 642, 644, 646, and 648, machine learning model may compare each of ratios 642, 644, 646, and 648 to an associated range of indicator values in ranges 650 to determine if the subject has or is likely to have the first condition or the second condition. In this depicted embodiment, an associated range of indicator values is associated with the same genes as the compared ratio (e.g., if a ratio is determined by dividing a first gene by a second gene, the range of indicator values is also determined using the first gene and the second gene).

In some embodiments, the ranges of indicator values are determined based on previously calculated ratios from sample received from previous subjects with the first condition and/or second condition. In some embodiments, the ranges of indicator values may span from the highest ratio to the lowest ratio calculated from samples received from previous subjects.

In some embodiments, only one ratio is calculated to determine if the subject has the first condition or the second condition. In some embodiments, a first ratio is calculated and determined to be inconclusive. In those embodiments, a second ratio or any number of ratios may be calculated after the first ratio is determined to be inconclusive.

After calculating ratios 642, 644, 646, and 648 and comparing ratios 642, 644, 646, and 648 to associated ranges of indicator values in ranges 650, one or more ratio indications indicating whether the subject has the first condition or the second condition may be generated.

Machine learning component 518 may then generate an indication 616 based on the classifier gene pairs 612 and the ratio indicators 614, where the indication 616 indicates whether the subject has, is likely to have, is unlikely to have, or does not have, the first condition, the second condition, both, or neither. In this depicted embodiment, the machine learning component 518 further provides ratio 618, which may include any number of ratios 642, 644, 646, and 648. In some embodiments, the machine learning component 518 does not provide the ratios 618.

Thus, by receiving expression levels 610 machine learning component 518 may classify each gene regarding whether the subject has, is likely to have, is unlikely to have, or does not have one or more conditions, may further calculate one or more ratios regarding that the subject has, is likely to have, is unlikely to have, or does not have the one or more conditions, and may generate an indication 616 to show whether the subject has, is likely to have, is unlikely to have, or does not have the one or more conditions based on the classifications and the ratios.

While four classifiers are shown for this depicted example, these classifiers are exemplary, and other classifiers may be used. For example, while the above description describes the classifiers as indicating a first condition, a second condition, both, or neither, classifiers may also only be associated with one condition or more be associated with more than two conditions.

While four ratios are shown for this depicted example, these ratios are exemplary and other ratios may be used. For example, while the above description describes four ratios being calculated, only one ratio may be calculated, or any number of ratios may be calculated.

As described above, in alternative embodiments, the each ratio may be compared to a respective single indicator value as opposed to a range of indicator values. In those embodiments, if the ratio is higher than the single indicator value, the machine learning model 604 may provide an indication that the subject has a first condition (e.g., atopic dermatitis) instead of a second condition (e.g., psoriasis). In some embodiments, if the ratio is higher than the single indicator value, the machine learning model 604 may provide an indication that the subject has a second condition (e.g., psoriasis) instead of a first condition (e.g., atopic dermatitis).

As described above with respect to FIGS. 2-5, in some embodiments, machine learning component 518 does not include machine learning model 602 (e.g., the first machine learning model as described with respect to FIGS. 2-5). In those embodiments, machine learning model 604 still receives the expression levels 610, determines ratios 642, 644, 646, and 648, compares ratios 642, 644, 646, and 648 to ranges 650, and provides ratio indications.

As described above with respect to FIGS. 2-5 and 7, in some embodiments, a ratio based on the received sample's gene expression levels is determined and compared to a single ratio to indicate whether or not the subject has a first condition (e.g., atopic dermatitis) instead of a second condition (e.g., psoriasis). In some embodiments, if the ratio based on the received sample is higher than the single ratio, the ratio indications 614 will indicate that the subject has the first condition instead of the second condition. In some embodiments, if the ratio based on the received sample is higher than the single ratio, the ratio indications 614 will indicate that the subject has the second condition instead of the first condition.

In some embodiments, machine learning component 518 may include another machine learning model as described above, which may receive ratio data and diagnoses data as feedback to adjust one or more indicator values.

FIG. 7 shows an example process by machine learning component 518. In this depicted example, machine learning component 518 shows includes machine learning model 702.

In this depicted embodiment, machine learning component receives expression levels 610. Expression levels 610 includes a plurality of expression levels of genes, where the expression levels of genes are determined based on a sample taken from a subject. The genes may include one or more of the IL13, IL4R, CCL17, CCL26, IL23A, IL22, S100A7, S100A8, S100A9, CXCL9, CXCL10, CXCL11, NOS2, IL17A, IL13R, CCL19, CCL27, TSLP, CCL18, IL23A, IL4R, and/or IL31 genes. As described above, these genes are exemplary, and others not listed may used instead of or in conjunction with the above genes.

In this depicted embodiment, machine learning model 702 receives expression levels 610 and determines ratio 720. The ratio 720 may be determined by dividing the normalized expression level of a first gene by the normalized expression level of second gene, by dividing the normalized expression level of a first gene by the sum of normalized expression levels of a second gene and a third gene, by dividing the sum of normalized expression levels of a first gene and a second gene by the sum of normalized expression levels of a third gene and a fourth gene, or by dividing the sum of normalized expression levels of a first gene and a second gene by a normalized expression ratio of a third gene. In some embodiments, a ratio may be determined by dividing the sum of normalized expression levels of four different genes by the sum of normalized expression ratios of four other genes, where the four other genes are different from the four different genes. In some embodiments, a ratio may be calculated by dividing a normalized expression level of one gene or a sum of normalized expression levels for any number of different genes by a normalized expression level of another gene or a sum of normalized expression levels for any number of other genes, wherein the other genes are not included in the different genes.

After determining ratio 720, machine learning model 702 may compare ratio 720 to indicator value 722 (as described above with respect to FIGS. 2-5). If ratio 720 is higher than indicator value 722, machine learning model 702 may provide a ratio indication 714 indicating that the subject has a first condition (e.g., atopic dermatitis) rather than a second condition (e.g., psoriasis). If ratio 720 is lower than indicator value 722, machine learning model 702 may provide a ratio indication 714 indicating that the subject has the second condition rather than the first condition.

Machine learning component 518 may then generate an indication 716 based on the classifier ratio indicator 714, where the indication 716 indicates whether the subject has or is likely to have the first condition instead of the second condition or the second condition instead of the first condition. In this depicted embodiment, the machine learning component 518 further provides ratio 720.

Thus, by receiving expression levels 610 machine learning component 518 may calculate ratio 720 and compare it indicator value 722 to determine if the subject has or is likely to have one condition rather than another condition, and may generate an indication 716 to show whether the subject has or is likely to have the one condition or another based on the comparison.

### Example System for Training One or More Machine Learning Models

FIG. 8 depicts an example computer system 800 for training a machine learning model to diagnose atopic dermatitis, psoriasis, or both, in a human subject. In this depicted example, system 500 includes server 510 and computing device 830.

In this depicted example, server 510 further includes receiving component 512, analyzing component 516, machine learning component 518, and database 520 as described with respect to FIG. 5.

In this depicted example, computing device 830 further includes UI component 832. UI component 832 may be configured to display indications received from the server 510 (e.g., indication 820) and may further be configured to receive user input (e.g., for feedback 840). In some embodiments, computing device is further configured to provide feedback 840.

In this depicted embodiments, computing device is configured to provide training data 810 to server 510. Training data 810 may include one or more gene expression levels for training one or more machine learning models of machine learning component 518 (e.g., the first machine learning model as described with respect to FIG. 5 or machine learning model 602 of FIG. 6). The gene expression levels may further be for training one or more machine learning models (e.g., the second machine learning model as described with respect to FIG. 5 or machine learning model 604 of FIG. 6) to determine one or more ratios and compare ratios to one or more ranges or a single ratio (as described with respect to FIGS. 2-5). In some embodiments, the training data 810 may be used by another machine learning model (as described above with respect to FIGS. 5-7) to determine one or more indicator values that ratios may be compared to.

Machine learning component 518 may provide an indication 820 based on the expression levels, where in the indication 820 indicates if a subject has, is likely to have, is unlikely to have, or does not have a first condition (e.g., atopic dermatitis) instead of a second condition (e.g., psoriasis), the second condition instead of the first condition, both, or neither. In some embodiments, the indication 820 may instead only indicate whether the subject has the first condition instead of the second condition after comparing the determined ratio to the single ratio.

Upon receiving the indication, the computing device 830 may provide feedback 840. In some embodiments, the feedback 840 may include user input to the computing device 830 through UI component 832. In some embodiments, the feedback 840 may include an updated indication, an updated ratio, or an updated classifier. For example, if the indication 820 indicates that, based on a determined ratio, the subject has a first condition instead of a second condition, the feedback 840 may provide an updated indication showing that the subject had the second condition instead of the first condition. Based on the feedback 840, the machine learning component 518 may adjust one or more parameters of the one or more machine learning models (such as the another machine learning model, machine learning models 602 and 604 of FIG. 6, or machine learning model 702 of FIG. 7), thereby training the one or more machine learning models.

While various embodiments of the present subject matter have been shown and described herein, it will be obvious to those skilled in the art that such embodiments are provided by way of example only. Numerous variations, changes, and substitutions may occur to those skilled in the art without departing from the subject matter described herein. It should be understood that various alternatives to the embodiments of the subject matter described herein may be employed.

### Example Method for Differentiating Atopic Dermatitis From Psoriasis

FIG. 9 depicts an example method 900 for determining if a subject has atopic dermatitis or psoriasis. In some embodiments, a skin sample from the subject may be analyzed to determine if the subject has atopic dermatitis or psoriasis (e.g., as described with respect to FIGS. 1-2 and 5-8). In some embodiments, the skin sample is lesional. In some embodiments, the skin sample is obtained using the methods described above using a non-invasive or semi-invasive technique (e.g., through use of adhesive patches or microneedles). In some embodiments, nucleic acids are isolated from the skin sample.

The method 900 begins at step 902 with receiving a plurality of expression levels, where the expression levels were derived from the skin sample that was obtained using the non-invasive or semi-invasive technique. The plurality of expression levels may be of one or more genes, where the one or more genes include any number of IL13, IL4R, CCL17, CCL26, IL23A, IL22, S100A7, S100A8, S100A9, CXCL9, CXCL10, CXCL 11, NOS2, IL17A, IL13R, CCL19, CCL27, TSLP, CCL18, IL23A, IL4R, and IL31. In some embodiments, the plurality of expression levels are normalized with respect to ACTB.

At step 904, a ratio between at least two of the plurality of expression of expression levels is calculated. In some embodiments, the ratio is calculated by a machine learning model (e.g., machine learning model 604 of FIG. 6 or machine learning model 702 of FIG. 7). In some embodiments, the ratio is determined by dividing a sum of an expression level of IL13 and an expression level of CCL17 by a sum of an expression level of NOS2 and an expression level of IL17A. In some embodiments, at least one classifier associated with at least one expression level of the plurality of expression levels is assigned to one or more genes. In some embodiments, the classifier can indicate if the expression level indicates if the subject has atopic dermatitis, psoriasis, both, or neither. In some embodiments, another machine learning model assigns the classifier (e.g., the machine learning model 602 of FIG. 6).

At step 906, the ratio is compared to a second ratio. In some embodiments, the second ratio is a ratio determined based on a plurality of previous expression levels by previous patients with atopic dermatitis or psoriasis. In some embodiments, the ratio may be higher or lower than the second ratio.

At step 908, an indication indicating whether the subject has atopic dermatitis or psoriasis is generated indicated based on comparing the ratio and the second ratio. In some embodiments, if the ratio is higher than the second ratio, the indication indicates that the subject has atopic dermatitis instead of psoriasis. In some embodiments, if the ratio is higher than the second ratio, the indication indicates that the subject has psoriasis instead of atopic dermatitis. In some embodiments, the indication is generated based further on the classifier. In the embodiments where the indication is generated also based on the classifier, the indication may indicate that the subject has atopic dermatitis instead of psoriasis, psoriasis instead of atopic dermatitis, has both, or has neither.

Accordingly, by receiving the plurality of expression levels, calculating the ratio, comparing the ratio, and generating the indication, subjects can be diagnosed as having either atopic dermatitis or psoriasis.

### Computer Systems

The present disclosure provides computer systems that are programmed to implement methods of the disclosure. FIG. 10 shows a computer system 1001 that is programmed or otherwise configured to generate indications showing if a subject has atopic dermatitis or psoriasis based on one or more calculated and/or compared ratios. The computer system 1001 can regulate various aspects of generating indications of the present disclosure, such as, for example, receiving a plurality of expression values associated with a subject, calculating one or more ratios, assigning one or more classifiers, comparing one or more ratios, or other aspects of the present disclosure. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., computing device 530 of FIG. 5). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, providing an alternate diagnosis of atopic dermatitis or psoriasis. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, allow the system 1001 to calculate and compare ratios as well as generate indications as described herein.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, my SQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Referring to FIG. 11, in a particular embodiment, an application provision system comprises one or more databases 1100 accessed by a relational database management system (RDBMS) 1110. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 1120 (such as Java servers, .NET servers, PHP servers, and the like) and one or more web servers 1130 (such as Apache, IIS, GWS and the like). The web server(s) optionally expose one or more web services via app application programming interfaces (APIs) 1140. Via a network, such as the Internet, the system provides browser-based and/or mobile native user interfaces.

Referring to FIG. 12, in a particular embodiment, an application provision system alternatively has a distributed, cloud-based architecture 1200 and comprises elastically load balanced, auto-scaling web server resources 1210 and application server resources 1220 as well synchronously replicated databases 1230.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of user information, gene expression levels, ratios, and classifiers. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### EXAMPLES

These examples are provided for illustrative purposes only and not to limit the scope of the claims provided herein.

### Example 1—Training a Machine Learning Model to Analyze Skin Samples From Patients With AD or PS

Epidermal skin samples were non-invasively collected from the lesional skin of the patients with moderate to severe AD (n=20) or moderate to severe psoriasis (n=20). RNA was isolated and analyzed by quantitative real-time PCR for the expression levels of twenty different genes. The expression levels were normalized based on ACTB being used as a housekeeping gene (e.g., to account for differences in sample input amounts).

A random forest machine learning model trained to receive gene expression levels and priorities genes to be evaluated as ratios of genes elevated in AD compared to genes elevated in severe psoriasis. The best identified ratio then compared the gene expression levels of CCL17, IL13, IL17A, and NOS2 by dividing the sum of normalized expression levels of CCL17 and IL13 by the sum of normalized expression levels for IL17A and NOS 2 to generate a ratio for each skin sample to be compared to an indicator value in order to determine if each patient has atopic dermatitis or psoriasis.

### Example 2—Using a Machine Learning Model to Analyze Skin Samples From Patients With AD or PS

Epidermal skin samples were non-invasively collected from the lesional skin of a patient not yet diagnosed with AD or PS. RNA was isolated and analyzed by quantitative real-time PCR for the expression levels of twenty different genes. The expression levels were normalized based on ACTB being used as a housekeeping gene.

The gene expression levels are provided to the machine learning model of Example 1. The machine learning model compares the gene expression levels of CCL17, IL13, IL17A, and NOS2 by dividing the sum of normalized expression levels of CCL17 and IL13 by the sum of normalized expression levels for IL17A and NOS2 to generate a ratio for the skin sample, where the ratio is then compared to an indicator value in the model.

### Example 3—Further Training A Machine Learning Model to Analyze Skin Samples From Patients With AD or PS

The patient of Example 2 is then diagnosed with having atopic dermatitis. The determined ratio of Example 2 is then provided to a regression machine learning model with an indicator showing that the patient was diagnosed as having atopic dermatitis. The regression machine learning model adjusts one or more parameters in response, as well as adjusting one or more associated indicator values to be provided as updated indicator values for use of the random forest model, thereby further training the machine learning model.

### Example 4—Training A Machine Learning Model to Analyze Skin Samples From Patients With AD or PS

Psoriasis and atopic dermatitis (AD) are two of the most prevalent chronic inflammatory skin diseases. Currently, diagnosis of psoriasis and AD is based on the combination of a skin exam and review of medical history. In some instances, the overlapping clinical characteristics and disease manifestations make it difficult to distinguish these two diseases, sometimes prompting a skin biopsy to look for the characteristic psoriatic histopathologic features. While effective, skin biopsies are invasive and have the potential for complications, especially in diseases characterized by abnormalities in the skin barrier and chronic S. aureus colonization. Here, we describe a non-invasive method to differentiate AD and psoriasis by comparing the expression of key genes involved in disease pathogenesis in AD and psoriasis. Epidermal skin samples were non-invasively collected from the lesional or nonlesional skin of the patients with moderate to severe AD (n=20) or moderate to severe psoriasis (n=20) using the DermTech Smart Sticker. RNA was isolated and analyzed by quantitative real-time PCR for the expression of IL-13, IL-23, IL-17A, S100A8, S100A9, CXCL9, CXCL10, CCL17 (TARC), CCL18 (PARC), CCL27 (Eotaxin-3), TLSP, and NOS2. The expression levels were normalized based on ACTB being used as a housekeeping gene.

A logistic regression machine learning model trained to receive gene expression levels and calculate various ratios based on the gene expression levels was provided the expression levels of the twenty different genes for each patient.

Dysregulation of IL-13, CCL17, IL-17A, and NOS2 exhibited the greatest differences between psoriasis and AD. Overall, this study demonstrates the potential utility of noninvasive skin sampling to differentiate AD and psoriasis based on a molecular signature from only four genes. The ability to distinguish these two disease conditions provides a valuable asset in the hands of physicians for clinical decision-making and can be utilized for the personalized treatment of AD and psoriasis patients.

### Example 5—Using a Machine Learning Model to Analyze Skin Samples From Patients With AD or PS

Epidermal skin samples were non-invasively collected from the lesional skin of a patient not yet diagnosed with AD or PS. RNA was isolated and analyzed by quantitative real-time PCR for the expression levels of twenty different genes. The expression levels were normalized based on ACTB being used as a housekeeping gene.

The gene expression levels are provided to the machine learning model of Example 1. The machine learning model compares the gene expression levels of IL13 and IL13R by dividing the normalized expression level of IL13 by the normalized expression level for IL13R to generate a ratio for the skin sample, where the ratio is then compared to a defined ratio in the model. The calculated ratio is determined to be above the defined ratio, indicating that the subject has psoriasis.

### Example 6—Further Training a Machine Learning Model to Analyze Skin Samples From Patients With Ad or Ps

The patient of Example 5 is then diagnosed with having atopic dermatitis. The determined ratio of example 5 is then provided to the regression machine learning model of Example 3 with an indicator showing that the patient was diagnosed as having psoriasis. The machine learning model adjusts one or more parameters in response, thereby further training the machine learning model.

What is claimed is:

1. A method for determining if a subject has atopic dermatitis or psoriasis, comprising:
receiving a plurality of expression levels, wherein the expression levels are derived from a skin sample obtained using a non-invasive or semi-invasive technique;
calculating a ratio between at least two of the plurality of expression levels;
determining whether the ratio is above or below an indicator value; and
generating an indication, wherein:
the indication indicates that the subject has a first condition if the ratio is above the indicator value,
the indication indicates that the subject has a second condition if the ratio is below the indicator value,
the first condition is different from the second condition,
the first condition is atopic dermatitis or psoriasis, and
the second condition is atopic dermatitis or psoriasis.

2. The method of claim 1, wherein the plurality of expression levels are determined by:
a. obtaining the skin sample;
b. isolating nucleic acids from the skin sample; and
c. measuring the plurality of expression levels.

3. The method of claim 1 or 2, wherein the plurality of expression levels are associated with a plurality of genes.

4. The method of claim 3, wherein the plurality of genes comprises IL13, IL4R, CCL17, CCL26, IL23A, IL22, S100A7, S100A8, S100A9, CXCL9, CXCL10, CXCL 11, NOS2, IL17A, IL13R, CCL19, CCL27, TSLP, CCL18, IL23A, IL4R, and IL31.

5. The method of claim 3 or 4, wherein the plurality of genes comprises IL13, CCL17, NOS2, and IL17A.

6. The method of any one of claims 1 to 5, wherein the ratio is determined by dividing a sum of an expression level of IL13 and an expression level of CCL17 by a sum of an expression level of NOS2 and an expression level of IL17A.

7. The method of any one of claims 1 to 6, wherein:
calculating the ratio between the at least two of the plurality of expression levels is performed by a machine learning model, and
determining whether the ratio is above or below the indicator value is performed by the machine learning model.

8. The method of any one of claims 1 to 7, wherein the indicator value is 1.

9. The method of any one of claims 1 to 8, wherein an AUC for the indicator value is at least 0.94.

10. The method of any one of claims 1 to 9, wherein the AUC for the indicator value is at least 0.83.

11. The method of any one of claims 1 to 10, further comprising assigning a classifier to at least one expression level of the plurality of expression levels.

12. The method of claim 11, wherein generating the indication is further based on the classifier.

13. The method of any one of claims 1 to 12, wherein the non-invasive technique comprises use of one or more adhesive patches or microneedles.

14. The method of any one of claims 1 to 13, wherein use of the one or more adhesive patches collects at least 1.5 mg of skin cells.

15. The method of any one of claims 1 to 14, wherein the skin sample comprises skin cells from the stratum corneum.

16. The method of any one of claims 1 to 15, wherein the skin sample is obtained from a lesional area of the subject.

17. The method of any one of claims 1 to 16, wherein the skin sample is stable for up to 10 days.

18. The method of claim 17, wherein the skin sample is stored at 10-30 degrees C.

19. The method of any one of claims 2 to 18, wherein the isolated nucleic acids comprise DNA and/or RNA.

20. The method of any one of claims 1 to 19, wherein the skin sample comprises one or more of epithelial acanthosis and mononuclear perivascular infiltrates.

21. The method of any one of claims 2 to 20, wherein the isolated nucleic acids are amplified prior to measuring biomarkers.

22. The method of any one of claims 2 to 21, wherein the measuring comprises detecting the plurality of expression levels of one or more target genes by measuring one or more biomarkers.

23. The method of claim 22, wherein the detecting gene expression levels comprises hybridizing one or more probes to nucleic acids corresponding to the one or more target genes.

24. The method of any one of claims 22 or 23, wherein the detecting gene expression levels comprises mass tagging and/or qPCR.

25. The method of any one of claims 22 to 24, wherein the one or more biomarkers comprises 2 or more target genes.

26. The method of any one of claims 22 to 25, wherein the one or more biomarkers comprises 5 or more target genes.

27. The method of any one of claims 22 to 26, wherein the one or more biomarkers comprises no more than 50 target genes.

28. The method of any one of claims 22 to 27, wherein the one or more target genes is differentially expressed in atopic dermatitis vs. psoriasis.

29. A system, comprising:
one or more processors; and
a memory comprising executable instructions which, when executed by the one or more processors, cause the system to:
receive a plurality of expression levels, wherein the expression levels are derived from a skin sample obtained using a non-invasive or semi-invasive technique;
calculate a ratio between at least two of the plurality of expression levels;
determine whether the ratio is above or below an indicator value; and
generate an indication, wherein:
the indication indicates that the subject has a first condition if the ratio is above the indicator value,
the indication indicates that the subject has a second condition if the ratio is below the indicator value,
the first condition is different from the second condition,
the first condition is atopic dermatitis or psoriasis, and
the second condition is atopic dermatitis or psoriasis.

30. The system of claim 29, wherein the plurality of expression levels are determined by:
a. obtaining the skin sample;
b. isolating nucleic acids from the skin sample; and
c. measuring the plurality of expression levels.

31. The system of claim 29 or 30, wherein the plurality of expression levels are associated with a plurality of genes.

32. The system of claim 31, wherein the plurality of genes comprises IL13, IL4R, CCL17, CCL26, IL23A, IL22, S100A7, S100A8, S100A9, CXCL9, CXCL10, CXCL 11, NOS2, IL17A, IL13R, CCL19, CCL27, TSLP, CCL18, IL23A, IL4R, and IL31.

33. The system of claim 31 or 32, wherein the plurality of genes comprises IL13, CCL17, NOS2, and IL17A.

34. The method of any one of claims 29 to 33, wherein:
calculating the ratio between the at least two of the plurality of expression levels is performed by a machine learning model, and
determining whether the ratio is above or below the indicator value is performed by the machine learning model.

35. The system of any one of claims 29 to 34, wherein the ratio is determined by dividing a sum of an expression level of IL13 and an expression level of CCL17 by a sum of an expression level of NOS2 and an expression level of IL17A.

36. The system of any one of claims 29 to 35, wherein the indicator value is 1.

37. The system of any one of claims 29 to 36, wherein an AUC for the indicator value is at least 0.94.

38. The system of any one of claims 29 to 37, wherein the AUC for the indicator value is at least 0.83.

39. The system of any one of claims 29 to 38, wherein the one or more processors are further configured to cause the system to assign a classifier to at least one expression level of the plurality of expression levels.

40. The system of claim 39, wherein one or more processors being configured to cause the system to generate the indication is further based on the classifier.

41. The system of any one of claims 29 to 40, wherein the non-invasive technique comprises use of one or more adhesive patches or microneedles.

42. The system of any one of claims 29 to 41, wherein use of the one or more adhesive patches collects at least 1.5 mg of skin cells.

43. The system of any one of claims 29 to 42, wherein the skin sample comprises skin cells from the stratum corneum.

44. The system of any one of claims 29 to 43, wherein the skin sample is obtained from a lesional area of the subject.

45. The system of any one of claims 29 to 44, wherein the skin sample is stable for up to 10 days.

46. The system of claim 45, wherein the skin sample is stored at 10-30 degrees C.

47. The system of any one of claims 30 to 46, wherein the isolated nucleic acids comprise DNA and/or RNA.

48. The system of any one of claims 29 to 47, wherein the skin sample comprises one or more of epithelial acanthosis and mononuclear perivascular infiltrates.

49. The system of any one of claims 30 to 48, wherein the isolated nucleic acids are amplified prior to measuring biomarkers.

50. The system of any one of claims 30 to 49, wherein the measuring comprises detecting the plurality of expression levels of one or more target genes by measuring one or more biomarkers.

51. The system of claim 50, wherein the detecting gene expression levels comprises hybridizing one or more probes to nucleic acids corresponding to the one or more target genes.

52. The system of any one of claims 50 or 51, wherein the detecting gene expression levels comprises mass tagging and/or qPCR.

53. The system of any one of claims 50 to 52, wherein the one or more biomarkers comprises 2 or more target genes.

54. The system of any one of claims 50 to 53, wherein the one or more biomarkers comprises 5 or more target genes.

55. The system of any one of claims 50 to 54, wherein the one or more biomarkers comprises no more than 50 target genes.

56. The system of any one of claims 50 to 55, wherein the one or more target genes is differentially expressed in atopic dermatitis vs. psoriasis.

57. A non-transitory, computer-readable medium comprising executable instructions, wherein a processor, when executing the executable instructions, performs a method for determining if a subject has atopic dermatitis or psoriasis, comprising:
receiving a plurality of expression levels, wherein the expression levels are derived from a skin sample obtained using a non-invasive or semi-invasive technique;
calculating a ratio between at least two of the plurality of expression levels;
determining whether the ratio is above or below an indicator value; and
generating an indication, wherein:
the indication indicates that the subject has a first condition if the ratio is above the indicator value,
the indication indicates that the subject has a second condition if the ratio is below the indicator value,
the first condition is different from the second condition,
the first condition is atopic dermatitis or psoriasis, and
the second condition is atopic dermatitis or psoriasis.

58. The non-transitory, computer-readable medium of claim 57, wherein the plurality of expression levels are determined by:
a. obtaining the skin sample;
b. isolating nucleic acids from the skin sample; and
c. measuring the plurality of expression levels.

59. The non-transitory, computer-readable medium of claim 57 or 58, wherein the plurality of expression levels are associated with a plurality of genes.

60. The non-transitory, computer-readable medium of claim 59, wherein the plurality of genes comprises IL13, IL4R, CCL17, CCL26, IL23A, IL22, S100A7, S100A8, S100A9, CXCL9, CXCL10, CXCL 11, NOS2, IL17A, IL13R, CCL19, CCL27, TSLP, CCL18, IL23Av2, IL4Rv2, and IL31.

61. The non-transitory, computer-readable medium of claim 59 or 60, wherein the plurality of genes comprises IL13, CCL17, NOS2, and IL17A.

62. The method of any one of claims 57 to 61, wherein:
calculating the ratio between the at least two of the plurality of expression levels is performed by a machine learning model, and
determining whether the ratio is above or below the indicator value is performed by the machine learning model.

63. The non-transitory, computer-readable medium of any one of claims 57 to 62, wherein the ratio is determined by dividing a sum of an expression level of IL13 and an expression level of CCL17 by a sum of an expression level of NOS2 and an expression level of IL17A.

64. The non-transitory, computer-readable medium of any one of claims 57 to 63, wherein the indicator value is 1.

65. The non-transitory, computer-readable medium of any one of claims 57 to 64, wherein an AUC for the indicator value is at least 0.94.

66. The non-transitory, computer-readable medium of any one of claims 57 to 65, wherein the AUC for the indicator value is at least 0.83.

67. The non-transitory, computer-readable medium of any one of claims 57 to 66, further comprising assigning a classifier to at least one expression level of the plurality of expression levels.

68. The non-transitory, computer-readable medium of claim 67, wherein generating the indication is further based on the classifier.

69. The non-transitory, computer-readable medium of any one of claims 57 to 68, wherein the non-invasive technique comprises use of one or more adhesive patches or microneedles.

70. The non-transitory, computer-readable medium of any one of claims 57 to 69, wherein use of the one or more adhesive patches collects at least 1.5 mg of skin cells.

71. The non-transitory, computer-readable medium of any one of claims 57 to 70, wherein the skin sample comprises skin cells from the stratum corneum.

72. The non-transitory, computer-readable medium of any one of claims 57 to 71, wherein the skin sample is obtained from a lesional area of the subject.

73. The non-transitory, computer-readable medium of any one of claims 57 to 72, wherein the skin sample is stable for up to 10 days.

74. The non-transitory, computer-readable medium of claim 73, wherein the skin sample is stored at 10-30 degrees C.

75. The non-transitory, computer-readable medium of any one of claims 58 to 74, wherein the isolated nucleic acids comprise DNA and/or RNA.

76. The non-transitory, computer-readable medium of any one of claims 57 to 75, wherein the skin sample comprises one or more of epithelial acanthosis and mononuclear perivascular infiltrates.

77. The non-transitory, computer-readable medium of any one of claims 57 to 76, wherein the isolated nucleic acids are amplified prior to measuring biomarkers.

78. The non-transitory, computer-readable medium of any one of claims 57 to 77, wherein the measuring comprises detecting the plurality of expression levels of one or more target genes by measuring one or more biomarkers.

79. The non-transitory, computer-readable medium of claim 78, wherein the detecting gene expression levels comprises hybridizing one or more probes to nucleic acids corresponding to the one or more target genes.

80. The non-transitory, computer-readable medium of any one of claims 78 or 79, wherein the detecting gene expression levels comprises mass tagging and/or qPCR.

81. The non-transitory, computer-readable medium of any one of claims 78 to 80, wherein the one or more biomarkers comprises 2 or more target genes.

82. The non-transitory, computer-readable medium of any one of claims 78 to 81, wherein the one or more biomarkers comprises 5 or more target genes.

83. The non-transitory, computer-readable medium of any one of claims 78 to 82, wherein the one or more biomarkers comprises no more than 50 target genes.

84. The non-transitory, computer-readable medium of any one of claims 78 to 83, wherein the one or more target genes is differentially expressed in atopic dermatitis vs. psoriasis.

85. A method useful for differentiating inflammatory skin diseases comprising:
a. obtaining a sample, wherein the sample is obtained using a non-invasive or semi-invasive technique from a subject;
b. isolating nucleic acids from the sample;
c. measuring one or more biomarkers from the isolated nucleic acids;
d. applying an algorithm to the one or more biomarkers to generate a pathology score; and
e. identifying the sample as having atopic dermatitis or psoriasis based on the pathology score.

86. The method of claim 85, wherein the non-invasive technique comprises use of one or more adhesive patches or microneedles.

87. The method of claim 85, wherein the sample comprises skin cells from the stratum corneum or blood.

88. The method of claim 85, wherein the sample comprises one or more of epidermal keratinocytes, T cells, dendritic cells, and melanocytes.

89. The method of any one of claim 86, wherein use of the one or more adhesive patches collects at least 1.5 mg of skin cells.

90. The method of any one of claims 85-89, wherein the sample is obtained from a lesional area of the subject.

91. The method of any one of claims 85-89, wherein the sample is obtained from a non-lesional area of the subject.

92. The method of any one of claims 85-91, wherein the sample is stable for up to 10 days.

93. The method of claim 92, wherein the sample is stored at 10-30 degrees C.

94. The method of any one of claims 85-93, wherein the isolated nucleic acids comprise DNA and/or RNA.

95. The method of any one of claims 85-94, wherein the sample comprises one or more of epithelial acanthosis and mononuclear perivascular infiltrates.

96. The method of any one of claims 85-95, wherein the isolated nucleic acids are amplified prior to measuring biomarkers.

97. The method of any one of claims 85-96, wherein the measuring comprises detecting gene expression levels of one or more target genes.

98. The method of claim 97, wherein the detecting gene expression levels comprises hybridizing one or more probes to nucleic acids corresponding to the one or more target genes.

99. The method of any one of claims 97-98, wherein the detecting gene expression levels comprises mass tagging and/or qPCR.

100. The method of any one of claims 85-99, wherein the one or more biomarkers comprises 2 or more target genes.

101. The method of any one of claims 85-100, wherein the one or more biomarkers comprises 5 or more target genes.

102. The method of any one of claims 85-101, wherein the one or more biomarkers comprises no more than 50 target genes.

103. The method of any one of claims 97-102, wherein the one or more target genes is differentially expressed in atopic dermatitis vs. psoriasis.

104. The method of any one of claims 97-103, wherein the one or more target genes are selected from the group consisting of TNF, LINC02571, HLA-C, HCP5, LCE3A-E, PSORS1C1, IFNG, IL12B, IL1B, NFKB1A, MUC22, and IL36RN.

105. The method of any one of claims 97-104, wherein the one or more target genes are selected from the group consisting of IL4-R, FLG, SPINK5, EMSY, PBX2, FLG-AS1, TSBP1, CRCT1, STAT3, CLDN1, NLRP10, IL18R1, TNFRSF6B, TNXB, TSLP/R, and STAT6.

106. The method of any one of claims 97-105, wherein the one or more target genes are selected from the group consisting of IL-13, IL-23, IL-17A, S100A8, S100A9, CXCL9, CXCL10, CCL17 (TARC), CCL18 (PARC), CCL27 (Eotaxin-3), TLSP, and NOS2.

107. The method of any one of claims 97-106, wherein the one or more target genes are selected from the group consisting of HLA-B, KPNA3, MGMT, R3GCC1L, STEAP-AS2, PRR5L, and IL-13.

108. The method of any one of claims 97-103, wherein the one or more target genes are selected from the group consisting of IL-4R, IL-13, CCL17/TARC, CCL26/Eotaxin 3, IL-17A, IL-22, IL-23A, NOS2, S100A8, S100A9, CXCL9, and CXCL10.

109. The method of any one of claims 97-103, wherein the one or more target genes are selected from the group consisting of IL-4R, IL-13, CCL17/TARC, and CCL26/Eotaxin 3.

110. The method of any one of claims 97-103, wherein the one or more target genes are selected from the group consisting of IL-17A, IL-22, IL-23A, NOS2, S100A8, S100A9, CXCL9, and CXCL10.

111. The method of any one of claims 97-103, wherein the one or more target genes are selected from the group consisting of IL-13, CCL17/TARC, NOS2, and IL-17A.

112. The method of any one of claims 85-111, wherein the algorithm comprises a ratio comparison of expression levels of two or more target genes.

113. The method of any one of claims 85-111, wherein the algorithm comprises a ratio comparison of expression levels of four or more target genes.

114. The method of any one of claims 85-111, wherein the algorithm comprises a ratio comparison of expression levels of no more than 50 target genes.

115. The method of any one of claims 85-114, wherein the algorithm comprises a ratio comparison of CCL17+IL-13 expression levels divided by NOS2+IL-17A expression levels.

116. The method of any one of claims 85-115, wherein a pathology score greater than 1 is indicative of atopic dermatitis in the sample.

117. The method of any one of claims 85-115, wherein a pathology score less than 1 is indicative of psoriasis in the sample.

118. The method of any one of claims 85-117, wherein the method comprises calculating an AUC.

119. The method of claim 118, wherein the AUC is at least 0.90.

120. The method of claim 118, wherein the AUC is at least 0.94.

121. The method of any one of claims 118-120, wherein the method comprises calculating an AUC.

122. The method of claim 121, wherein the AUC is at least 0.94 and the p-value is less than 0.001.

123. The method of claim 121, wherein the AUC is at least 0.94 and the p-value is less than 0.0001.

124. The method of any one of claims 85-123, wherein the identifying the sample as having atopic dermatitis is based on a pathology score >0.9.

125. The method of any one of claims 85-123, wherein the identifying the sample as having psoriasis is based on a pathology score <0.9.

126. The method of any one of claims 85-123, wherein the identifying the sample as having atopic dermatitis is based on a pathology score >1.0.

127. The method of any one of claims 85-123, wherein the identifying the sample as having psoriasis is based on a pathology score <1.0.

128. The method of any one of claims 85-127, wherein the sample is obtained from a subject suspected as having moderate to severe atopic dermatitis or moderate to severe psoriasis.

129. The method of any one of claims 85-128, wherein the pathology score further identifies a disease subtype and/or a disease endotype.

130. The method of any one of claims 85-129, wherein the method further comprises administering a treatment specific to atopic dermatitis or psoriasis based on the identification of the sample as having atopic dermatitis or psoriasis and/or the pathology score.

131. The method of any one of claims 85-130, wherein the subject is a human subject.

132. A system for differentiating inflammatory skin diseases comprising:
a. a sample device configured to extract a sample from a subject;
b. a sample processing device configured to extract nucleic acids and measure one or more biomarkers associated with the nucleic acids;
c. at least one computer processor configured to (1) receive data comprising the one or more biomarkers and (2) execute an algorithm to process the data and output a pathology score; and
d. a communication module for transmission of the pathology score or a visual interface configured to display the pathology score.

133. The system of claim 132, wherein the algorithm is configured to generate the pathology score from a ratio of one or more gene expression levels.

134. The system of any one of claims 132-133, wherein the algorithm is configured to generate the pathology score by:
a. adding expression levels of a first and a second target gene to generate a first value;
b. adding expression levels of a third and a fourth target gene to generate a second value; and
c. dividing the first value by the second value to generate the pathology score.

135. The system of any one of claims 132-134, wherein increased expression of the first target gene and/or the second target gene are associated with atopic dermatitis.

136. The system of any one of claims 132-135, wherein increased expression of the third target gene and/or the fourth target gene are associated with psoriasis.

137. The system of any one of claims 132-136, wherein the pathology score greater than 1 is indicative of atopic dermatitis in the sample.

138. The system of any one of claims 132-136, wherein the pathology score less than 1 is indicative of psoriasis in the sample.

139. The system of any one of claims 132-138, wherein the first target gene and the second target gene are independently selected from the group consisting of IL-4R, IL-13, CCL17/TARC, and CCL26/Eotaxin 3.

140. The system of any one of claims 132-138, wherein the third target gene and the fourth target gene are independently selected from the group consisting of IL-17A, IL-22, IL-23A, NOS2, S100A8, S100A9, CXCL9, and CXCL10.

141. The system of any one of claims 132-138, wherein the first target gene and the second target gene are IL-13 and CCL17/TARC, respectively.

142. The system of any one of claims 132-138, wherein third target gene and the fourth target gene are IL-17A and NOS2, respectively.

143. The system of any one of claims 132-142, wherein the sample processing device comprises a DNA sequencer, a qPCR instrument, and/or a mass array instrument.

144. The system of any one of claims 132-143 wherein the subject is a human subject.

145. A composition comprising:
a. one or more target nucleic acid molecules derived from a non-invasive sample obtained from a subject; and
b. one or more probes configured to bind to the one or more target nucleic acid molecules, wherein the one or more target nucleic acid molecules correspond to one or more target genes, and wherein the one or more target genes is differentially expressed in atopic dermatitis vs. psoriasis.

146. The composition of claim 145, wherein the target nucleic acid molecules comprises DNA and/or RNA.

147. The composition of claim 146, wherein the DNA target nucleic acid molecule is a cDNA molecule.

148. The composition of any one of claims 145-147, wherein at least one of the one or more probes is hybridized to the one or more target nucleic acid molecules.

149. The composition of any one of claims 145-148, wherein the one or more target nucleic acid molecules corresponds to one or more target gene selected from the group consisting of IL-4R, IL-13, CCL17/TARC, CCL26/Eotaxin 3, IL-17A, IL-22, IL-23A, NOS2, S100A8, S100A9, CXCL9, and CXCL10.

150. The composition of any one of claims 145-148, wherein the one or more target nucleic acid molecules corresponds to one or more target gene selected from the group consisting of IL-13, CCL17/TARC, NOS2, and IL-17A.

151. The composition of any one of claims 145-150, wherein the one or more probes comprises a reporter moiety.

152. The composition of any one of claims 145-151 wherein the reporter moiety comprises a fluorescent label or a mass label.

153. The composition of any one of claims 145-152, wherein the composition further comprises at least one primer specific to the one or more target nucleic acid molecules.

154. The composition of any one of claims 145-153, wherein the composition further comprises at least one polymerase.

155. The composition of any one of claims 145-154, wherein the subject is a human subject.

156. The composition of claim 155, wherein the human subject is suspected of having an inflammatory disease.

157. The composition of any one of claims 145-156, wherein the inflammatory disease is atopic dermatitis or psoriasis.

158. The composition of any one of claims 145-157, wherein the atopic dermatitis or the psoriasis is moderate to severe.

159. The composition of any one of claims 145-158, wherein the inflammatory disease is characterized by abnormalities in the skin barrier and/or chronic S. aureus colonization.

160. The composition of any one of claims 145-159, wherein the sample is an epidermal skin sample.

161. The composition of any one of claims 145-160, wherein the sample is a lesional skin sample.

162. The composition of any one of claims 145-160, wherein the sample is a non-lesional skin sample.

163. The composition of any one of claims 145-162, wherein the non-invasive sample is collected using one or more adhesive patches.

164. The composition of any one of claims 145-163, wherein the one or more adhesive patches is a DermTech SmartSticker™. 165. The composition of any one of claims 145-164, wherein the differential expression of the one or more target genes provides a molecular signature indicative of atopic dermatitis or psoriasis.

166. A composition comprising:
a) a buffer;
b) an adhesive patch or portion of an adhesive patch; and
c) an epidermal skin sample comprising one or more target molecules.

167. The composition of claim 166, wherein the one or more target molecules is selected from the group consisting of a protein, RNA, DNA, and lipid.

168. The composition of any one of claims 166-167. wherein the epidermal skin sample comprises at least 1.5 mg of stratum corneum tissue.

169. The composition of any one of claims 166-168, wherein the epidermal skin sample is a lesional skin sample.

170. The composition of any one of claims 166-168, wherein the epidermal skin sample is a non-lesional skin sample.

171. The composition of any one of claims 166-170 wherein the composition does not comprise a fixative reagent.

172. The composition of any one of claims 166-171, wherein the composition is stable for up to 10 days.

173. The composition of any one of claims 166-172, wherein the epidermal skin sample comprises one or more cell types selected from the group consisting of keratinocytes, T-cells, dendritic cells, and melanocytes.

174. The composition of any one of claims 166-173, wherein the one or more target molecules is a target nucleic acid molecule.

175. The composition of any one of claims 166-174, wherein the target nucleic acid molecule is an RNA molecule.

176. The composition of claim 175, wherein the target nucleic acid molecule is a cDNA molecule.

177. The composition of any one of claims 166-176, wherein the RNA or cDNA molecule corresponds to one or more genes selected from the group consisting of IL-4R, IL-13, CCL17/TARC, CCL26/Eotaxin 3, IL-17A, IL-22, IL-23A, NOS2, S100A8, S100A9, CXCL9, and CXCL10.

178. The composition of any one of claims 166-176, wherein the RNA or cDNA molecule corresponds to one or more genes selected from the group consisting of IL-13, CCL17/TARC, IL-17A, and NOS2.

179. The composition of any one of claims 166-178, wherein the epidermal skin sample is from a region of the skin exhibiting histopathology indicative of epithelial acanthosis and/or mononuclear perivascular infiltrate.

180. The composition of any one of claims 166-179, wherein the target molecule is associated with a microbiome.

181. The composition of any one of claims 166-180, wherein the epidermal skin sample is obtained from a human subject.

182. The composition of any one of claims 166-181, wherein the human subject is suspected of having an inflammatory disease.

183. The composition of any one of claims 166-182, wherein the inflammatory disease is atopic dermatitis or psoriasis.
